(19) United States
(12) Patent Application Publication
(10) Pub. No.: US 2017/0355740 A1
(43) Pub. Date: Dec. 14, 2017

(54) METHODS OF USING FIBROBLAST GROWTH FACTOR (FGF) 1 WITH MUTATION IN THE HEPARIN BINDING DOMAIN AND METHODS TO REDUCE BLOOD GLUCOSE

(71) Applicant: Salk Institute for Biological Studies, La Jolla, CA (US)

(72) Inventors: Ronald M. Evans, La Jolla, CA (US); Michael Downes, San Diego, CA (US); Annette Atkins, San Diego, CA (US); Ruth T. Yu, La Jolla, CA (US)

(73) Assignee: Salk Institute for Biological Studies, La Jolla, CA (US)

(21) Appl. No.: 15/681,632

(22) Filed: Aug. 21, 2017

Related U.S. Application Data
(63) Continuation of application No. PCT/US2016/028365, filed on Apr. 20, 2016.
(60) Provisional application No. 62/149,823, filed on Apr. 20, 2015.

(51) Int. Cl. C07K 14/50; A61K 47/64; C12Q 1/54; C12N 15/85; A61K 48/00; A61K 38/00; C12N 15/00

(52) U.S. Cl. CPC C07K 14/503; C12Q 1/54; A61K 47/6425; C12N 15/00; A61K 48/00; A61K 38/00; C12N 2015/8518

(57) ABSTRACT

The present disclosure provides FGF1 mutant proteins having one or more mutations in the heparin binding domain. Such mutants may also have an N-terminal deletion, point mutation(s), or combinations thereof. In some examples, the mutant FGF1 proteins have reduced mitogenic activity. Also provided are nucleic acid molecules that encode such proteins, and vectors and cells that include such nucleic acids. The disclosed FGF1 mutants can reduce blood glucose in a mammal, and in some examples are used to treat a metabolic disorder.

### CROSS REFERENCE TO RELATED APPLICATIONS

This application is a continuation of International Application No. PCT/US2016/028365, filed Apr. 20, 2016, which was published in English under PCT Article 21(2), which in turn which claims priority to U.S. Provisional Application No. 62/149,823, filed on Apr. 20, 2015, both herein incorporated by reference.

### ACKNOWLEDGMENT OF GOVERNMENT SUPPORT

This invention was made with government support under Grant Nos. DK057978, DK090962, HL088093, HL105278, and ES010337 awarded by The National Institutes of Health. The government has certain rights in the invention.

### FIELD

This application provides mutated FGF1 proteins, nucleic acids encoding such proteins, and methods of their use, for example to reduce blood glucose and/or to treat a metabolic disease.

### BACKGROUND

Type 2 diabetes and obesity are leading causes of mortality and are associated with the Western lifestyle, which is characterized by excessive nutritional intake and lack of exercise. A central player in the pathophysiology of these diseases is the nuclear hormone receptor (NHR) PPARγ, a lipid sensor and master regulator of adipogenesis. PPARγ is also the molecular target for the thiazolidinedione (TZD)-class of insulin sensitizers, which command a large share of the current oral anti-diabetic drug market. However, there are numerous side effects associated with the use of TZDs such as weight gain, liver toxicity, upper respiratory tract infection, headache, back pain, hyperglycemia, fatigue, sinusitis, diarrhea, hypoglycemia, mild to moderate edema, and anemia. Thus, the identification of new insulin sensitizers is needed.

### SUMMARY

It is shown herein that mutants of fibroblast growth factor (FGF) 1 that affect its interaction with heparan sulfate influence the duration of its glucose lowering effect. In addition, the introduction of these mutations into stabilized or FGFR1-targeted FGF-1 analogs can extend their glucose lowering effects in diabetic mice for up to 2 weeks from a single injection. Mutations that reduce heparan sulfate binding (e.g., K112D, K113Q, K118V) reduce the duration of the glucose lowering actions of FGF-1 analogs, while conversely (see U.S. patent application Ser. No. 14/520,178, herein incorporated by reference in its entirety), mutations that enhance binding (e.g., S116R) extend the duration of the effects in diabetic mice. Based on these observations, the duration of the glucose lowering effect of FGF1 can be manipulated through targeted mutations of amino acids that bind to heparan sulfate, or are close in 3 dimensional space to the heparan sulfate binding site. Based on these observations, methods for reducing blood glucose in a mammal, for example to treat a metabolic disease, are disclosed. Such FGF1 mutants can further have an N-terminal truncation, additional point mutation(s), or combinations thereof, for example to reduce the mitogenic activity and/or increase the thermostability (e.g., by introducing a mutation at C117, such as C117V) of the FGF1 protein (e.g., relative to a native FGF1 protein). Such FGF1 mutants can be used alone or in combination with other agents, such as other glucose reducing agents, such as thiazolidinediones or insulin. In some examples, use of the disclosed mutant FGF1 proteins result in one or more of: reduction in triglycerides, decrease in insulin resistance, reduction of hyperinsulinemia, increase in glucose tolerance, reduction of food intake, or reduction of hyperglycemia in a mammal.

Provided herein are mutated FGF1 proteins containing one or more mutations that affect the ability of FGF1 to interact with heparan sulfate, and thus influence the duration of its glucose lowering effect. In one example, a mutant FGF1 protein has increased heparan sulfate binding affinity, thereby extending the duration of its functional activity. In some examples, a mutated FGF1 protein includes a mutation at S116, such as S116R, which can have increased heparan sulfate binding affinity relative to a native FGF1 protein (e.g., SEQ ID NO: 5), such as an increase of at least 20%, at least 30%, at least 40%, at least 50%, at least 75%, at least 80%, at least 90%, at least 100%, or at least 200%. In some examples, such FGF1 mutants further include deletion of an N-terminal portion of FGF1, point mutation(s) (such as amino acid substitutions, deletions, additions, or combinations thereof), or combinations of N-terminal deletions and point mutation(s). In some examples, such additional mutations reduce the mitogenicity relative to mature FGF1 (e.g., SEQ ID NO: 5), such as a reduction of at least 20%, at least 50%, at least 75% or at least 90%. In some examples, such additional mutations increase the thermostability relative to mature FGF1 (e.g., SEQ ID NO: 5), such as an increase of at least 20%, at least 50%, at least 75%, at least 90%, at least 100%, or at least 200%. In some examples, the mutant FGF1 protein containing a mutation at S116 is a truncated version of the mature protein (e.g., SEQ ID NO: 5), which can include for example deletion of at least 5, at least 6, at least 10, at least 11, at least 12, at least 13, or at least 20 consecutive N-terminal amino acids. In some examples, the mutant FGF1 protein containing a mutation at S116 includes further mutations, such as one containing at least 1, at least 2, at least 3, at least 4, at least 5, at least 6, at least 7, at least 8, at least 9 or at least 10 additional amino acid substitutions (such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or 41 substitutions), such as one or more of those shown in Table 1. In some examples, the mutant FGF1 protein containing a mutation at S116 further includes both an N-terminal truncation and one or more additional point mutations. In some examples, the mutant FGF1 protein containing a mutation at S116 includes at least 120 consecutive amino acids from amino acids 5-141 of FGF1 (e.g., of SEQ ID NOS: 2 or 4), (which in some examples can include 1-20 point mutations, such as substitutions, deletions, and/or additions).

Also provided are isolated nucleic acid molecules encoding the disclosed mutant FGF1 proteins. Vectors and cells that include such nucleic acid molecules are also provided.

Methods of using the disclosed mutant FGF1 proteins (or nucleic acid molecules encoding such) containing a mutation at S116 are provided, such as a mutated mature FGF1 protein having a mutation at S116 and a deletion of at least six contiguous N-terminal amino acids (and in some examples at least one more point mutation), for example to reduce or eliminate mitogenic activity. In some examples the methods include administering a therapeutically effective amount of a disclosed mutant FGF1 protein (or nucleic acid molecules encoding such) to reduce blood glucose in a mammal, such as a decrease of at least 5%, at least 10%, at least 25%, at least 50%, or at least 75%. In some examples the methods include administering a therapeutically effective amount of a disclosed mutant FGF1 protein (or nucleic acid molecules encoding such) to treat a metabolic disease in a mammal. Exemplary metabolic diseases that can be treated with the disclosed methods include, but are not limited to: diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), and cardiovascular diseases (e.g., hypertension). In some examples, one or more of these diseases are treated simultaneously with the disclosed FGF1 mutants. Also provided are methods of reducing fed and fasting blood glucose, improving insulin sensitivity and glucose tolerance, reducing systemic chronic inflammation, ameliorating hepatic steatosis in a mammal, reducing food intake, or combinations thereof, by administering a therapeutically effective amount of a disclosed mutant FGF1 protein (or nucleic acid molecules encoding such).

The foregoing and other objects and features of the disclosure will become more apparent from the following detailed description, which proceeds with reference to the accompanying figures.

### BRIEF DESCRIPTION OF THE DRAWINGS

FIG. 1 is a bar graph showing the effects of FGF1 analogs on blood glucose. Ob/ob mice were injected subcutaneously with vehicle (PBS) or the FGF1 analog shown in SEQ ID NO: 11 (0.5 mg/kg) and blood glucose levels recorded at indicated times. Blood glucose levels are expressed as percentage of initial glucose.

FIGS. 2A and 2B are bar graphs showing the effect of FGF1 analogs on (A) blood glucose levels and (B) food intake. Ob/ob mice were injected subcutaneously with vehicle (PBS) or an FGF1 analog (SEQ ID NOS: 12, 13, or 14) (0.5 mg/kg) and blood glucose levels (A) and food intake (B) determined. Blood glucose levels are expressed as percentage of initial glucose.

FIGS. 3A and 3B are bar graphs showing the effect of FGF1 analogs on (A) blood glucose levels and (B) food intake. Ob/ob mice were injected subcutaneously with vehicle (PBS) or an FGF1 analog (SEQ ID NOS: 12, 15, 16, 17, or 18) (0.5 mg/kg) and blood glucose levels (A) and food intake (B) determined. Blood glucose levels are expressed as percentage of initial glucose.

FIGS. 4A-4C are bar graphs showing the effect of FGF1 analog (A) Salk_061 (SEQ ID NO: 19), (B) Salk_066 (SEQ ID NO: 21), or (C) Salk_067 (SEQ ID NO: 22), on blood glucose levels. Ob/ob mice were injected subcutaneously with vehicle (PBS) or an FGF1 analog (SEQ ID NOS: 19, 20, 21, or 22) (0.5 mg/kg) and blood glucose levels determined. Blood glucose levels are expressed as percentage of initial glucose.

FIGS. 5A-5C are graphs showing the effect of FGF1 analogs on (A) and (B) blood glucose levels and (C) food intake. Ob/ob mice were injected subcutaneously with vehicle (PBS) or an FGF1 analog (SEQ ID NOS: 12 or 20) (0.5 mg/kg) and blood glucose levels (A) and food intake (B) determined. Blood glucose levels are expressed as percentage of initial glucose.

FIGS. 6A-6B are a series of graphs showing the blood glucose lowering effects of (A) Salk_052 (SEQ ID NO: 24), (B) Salk_073 (SEQ ID NO: 23). An artificial disulfide bond was engineered between amino acid positions 66 and 83.

FIG. 7 is a graph showing a functional assay (mitogenic/cell survival) for WT FGF-1 and S116R mutant protein quantified by radioactive 3H-thimidine incorporation/CPM. The protein concentration (pg/mL) is plotted as log 10 scale. 3H-thimidine incorporation at 0 pg/mL protein concentration is indicated by data points on the ordinate. Error bars are SEM (standard error of the measurement).

FIG. 8 shows an alignment between different mammalian wild-type FGF1 sequences (human (SEQ ID NO: 2), gorilla (SEQ ID NO: 79), chimpanzee (SEQ ID NO: 80), canine (SEQ ID NO: 81), feline (SEQ ID NO: 82), and mouse (SEQ ID NO: 4)). Such an alignment can be routinely generated in the art, and can be used to make the mutations provided herein to any FGF1 sequence of interest.

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing generated on Aug. 17, 2017 (74.5 kb) and submitted herewith is herein incorporated by reference.

SEQ ID NOS: 1 and 2 provide an exemplary human FGF1 nucleic acid and protein sequences, respectively. Source: GenBank Accession Nos: BC032697.1 and AAH32697.1.

SEQ ID NOS: 3 and 4 provide an exemplary mouse FGF1 nucleic acid and protein sequences, respectively. Source: GenBank Accession Nos: BC037601.1 and AAH37601.1.

SEQ ID NO: 5 provides an exemplary mature form of FGF1 (140 aa, sometimes referred to in the art as FGF1 15-154).

SEQ ID NOS: 6-9 provide exemplary mature forms of FGF1 with different N-terminal deletions.

SEQ ID NO: 10 provides a coding sequence for SEQ ID NO: 6.

SEQ ID NO: 11 provides an exemplary mature form of FGF1 with a point mutation (S116R) to increase binding to heparan sulfate.

SEQ ID NO: 12 provides an exemplary mature form of FGF1 with a point mutation (C117V) (Salk_014) to increase pharmacological stability.

SEQ ID NO: 13 provides an exemplary mature form of FGF1 with four point mutations (K12V, N95V, S116R, C117V) (Salk_050) to evaluate the combined effects of reduced mitogenicity, increased heparan sulfate binding affinity and improved pharmacological stability.

SEQ ID NO: 14 provides an exemplary N-terminally truncated form of FGF1 with two point mutations (S116R, C117V) (Salk_051) to evaluate the combined effects of increased heparan sulfate binding affinity and improved pharmacological stability.

SEQ ID NO: 15 provides an exemplary mature form of FGF1 with four point mutations (K12V, N95T, S116R, C117V) (Salk_053) to evaluate the combined effects of reduced mitogenicity, altered receptor binding affinity and/or specificity, increased heparan sulfate binding affinity and improved pharmacological stability.

SEQ ID NO: 16 provides an exemplary mature form of FGF1 with three point mutations (Y55A, S116R, C117V) (Salk_054) to evaluate the combined effects of altered receptor binding affinity and/or specificity, increased heparan sulfate binding affinity and improved pharmacological stability.

SEQ ID NO: 17 provides an exemplary mature form of FGF1 with three point mutations (Y55W, S116R, C117V) (Salk_055) to evaluate the combined effects of altered receptor binding affinity and/or specificity, increased heparan sulfate binding affinity and improved pharmacological stability.

SEQ ID NO: 18 provides an exemplary mature form of FGF1 with three point mutations (E87H, S116R, C117V) (Salk_056) to evaluate the combined effects of altered receptor binding affinity and/or specificity, increased heparan sulfate binding affinity and improved pharmacological stability.

SEQ ID NO: 19 provides an exemplary N-terminally truncated form of FGF1 with three point mutations (R35E, S116R, C117V) (Salk_061) to evaluate the combined effects of reduced mitogenicity, increased heparan sulfate binding affinity and improved pharmacological stability.

SEQ ID NO: 20 provides an exemplary mature form of FGF1 with three point mutations (E49A, S116R, C117V) (Salk_065) to evaluate the combined effects of altered receptor binding affinity and/or specificity, increased heparan sulfate binding affinity and improved pharmacological stability.

SEQ ID NO: 21 provides an exemplary N-terminally truncated form of FGF1 with five point mutations (K12V, Y94V, N95V, S116R, C117V) (Salk_066) to evaluate the combined effects of altered receptor binding affinity and/or specificity, reduced mitogenicity, increased heparan sulfate binding affinity and improved pharmacological stability.

SEQ ID NO: 22 provides an exemplary N-terminally truncated form of FGF1 with four point mutations (K12V, N95V, S116R, C117V) (Salk_067) to evaluate the combined effects of altered receptor binding affinity and/or specificity, reduced mitogenicity, increased heparan sulfate binding affinity and improved pharmacological stability.

SEQ ID NO: 23 provides an exemplary N-terminally truncated form of FGF1 with five point mutations (K12V, A66C, N95V, S116R, and C117V, wherein numbering refers to SEQ ID NO: 5).

SEQ ID NO: 24 provides an exemplary N-terminally truncated form of FGF1 with four point mutations (K12V, N95V, S116R, and C117V, wherein numbering refers to SEQ ID NO: 5).

SEQ ID NO: 25 provides an exemplary C-terminal FGF21 protein sequence (P¹⁶⁸-S²⁰⁹hFGF21^{C-tail}). This fragment can be attached at its N-terminus to the C-terminus of any FGF1 mutant provided herein to generate an FGF1/FGF21 chimera.

SEQ ID NO: 26 provides an exemplary C-terminal FGF19 protein sequence (L¹⁶⁹-K²¹⁶ h FGF19C-tail). This fragment can be attached at its N-terminus to the C-terminus of any FGF1 mutant provided herein to generate an FGF1/FGF19 chimera.

SEQ ID NO: 27 provides an exemplary β-Klotho binding protein dimer sequence (C2240) that can be attached at its N- or C-terminus directly or indirectly to any of the FGF1 mutants provided herein to generate a chimeric protein.

SEQ ID NO: 28 provides an exemplary β-Klotho binding protein sequence that can be attached at its N- or C-terminus directly or indirectly to any of the FGF1 mutants provided herein to generate a chimeric protein.

SEQ ID NO: 29 provides an exemplary β-Klotho binding protein sequence that can be attached at its N- or C-terminus directly or indirectly to any of the FGF1 mutants provided herein to generate a chimeric protein. In addition, it can be linked to SEQ ID NO: 28 via a linker and then the resulting chimera attached at its N- or C-terminus directly or indirectly to any of the FGF1 mutants provided herein to generate a chimeric protein.

SEQ ID NO: 30 provides an exemplary β-Klotho binding protein sequence that can be attached at its N- or C-terminus directly or indirectly to any of the FGF1 mutants provided herein to generate a chimeric protein. In addition, it can be linked to SEQ ID NO: 28 via a linker and then the resulting chimera attached at its N- or C-terminus directly or indirectly to any of the FGF1 mutants provided herein to generate a chimeric protein.

SEQ ID NO: 31 provides an exemplary β-Klotho binding protein sequence that can be attached at its N- or C-terminus directly or indirectly to any of the FGF1 mutants provided herein to generate a chimeric protein. In addition, it can be linked to SEQ ID NO: 28 via a linker and then the resulting chimera attached at its N- or C-terminus directly or indirectly to any of the FGF1 mutants provided herein to generate a chimeric protein.

SEQ ID NO: 32 provides an exemplary β-Klotho binding protein sequence that can be attached at its N- or C-terminus directly or indirectly to any of the FGF1 mutants provided herein to generate a chimeric protein. In addition, it can be linked to SEQ ID NO: 28 via a linker and then the resulting chimera attached at its N- or C-terminus directly or indirectly to any of the FGF1 mutants provided herein to generate a chimeric protein.

SEQ ID NO: 33 provides an exemplary β-Klotho binding protein sequence that can be attached at its N- or C-terminus directly or indirectly to any of the FGF1 mutants provided herein to generate a chimeric protein. In addition, it can be linked to SEQ ID NO: 28 via a linker and then the resulting chimera attached at its N- or C-terminus directly or indirectly to any of the FGF1 mutants provided herein to generate a chimeric protein.

SEQ ID NO: 34 provides an exemplary β-Klotho binding protein sequence can be attached at its N- or C-terminus directly or indirectly to any of the FGF1 mutants provided herein to generate a chimeric protein. In addition, it can be linked to SEQ ID NO: 28 via a linker and then the resulting chimera attached at its N- or C-terminus directly or indirectly to any of the FGF1 mutants provided herein to generate a chimeric protein.

SEQ ID NO: 35 provides an exemplary β-Klotho binding protein sequence can be attached at its N- or C-terminus directly or indirectly to any of the FGF1 mutants provided herein to generate a chimeric protein. In addition, it can be linked to SEQ ID NO: 28 via a linker and then the resulting chimera attached at its N- or C-terminus directly or indirectly to any of the FGF1 mutants provided herein to generate a chimeric protein.

SEQ ID NO: 36 provides an exemplary β-Klotho binding protein sequence can be attached at its N- or C-terminus directly or indirectly to any of the FGF1 mutants provided herein to generate a chimeric protein. In addition, it can be linked to SEQ ID NO: 28 via a linker and then the resulting chimera attached at its N- or C-terminus directly or indirectly to any of the FGF1 mutants provided herein to generate a chimeric protein.

SEQ ID NO: 37 provides an exemplary β-Klotho binding protein sequence that can be attached at its N- or C-terminus directly or indirectly to any of the FGF1 mutants provided herein to generate a chimeric protein.

SEQ ID NO: 38 provides an exemplary β-Klotho binding protein sequence that can be attached at its N- or C-terminus directly or indirectly to any of the FGF1 mutants provided herein to generate a chimeric protein.

SEQ ID NO: 39 provides an exemplary β-Klotho binding protein sequence that can be attached at its N- or C-terminus directly or indirectly to any of the FGF1 mutants provided herein to generate a chimeric protein.

SEQ ID NO: 40 provides an exemplary β-Klotho binding protein sequence that can be attached at its N- or C-terminus directly or indirectly to any of the FGF1 mutants provided herein to generate a chimeric protein.

SEQ ID NO: 41 provides an exemplary β-Klotho binding protein sequence that can be attached at its N- or C-terminus directly or indirectly to any of the FGF1 mutants provided herein to generate a chimeric protein.

SEQ ID NO: 42 provides an exemplary β-Klotho binding protein sequence that can be attached at its N- or C-terminus directly or indirectly to any of the FGF1 mutants provided herein to generate a chimeric protein.

SEQ ID NO: 43 provides an exemplary β-Klotho binding protein sequence that can be attached at its N- or C-terminus directly or indirectly to any of the FGF1 mutants provided herein to generate a chimeric protein.

SEQ ID NO: 44 provides an exemplary β-Klotho binding protein sequence that can be attached at its N- or C-terminus directly or indirectly to any of the FGF1 mutants provided herein to generate a chimeric protein.

SEQ ID NO: 45 provides an exemplary β-Klotho binding protein sequence that can be attached at its N- or C-terminus directly or indirectly to any of the FGF1 mutants provided herein to generate a chimeric protein.

SEQ ID NO: 46 provides an exemplary β-Klotho binding protein sequence that can be attached at its N- or C-terminus directly or indirectly to any of the FGF1 mutants provided herein to generate a chimeric protein.

SEQ ID NO: 47 provides an exemplary β-Klotho binding protein sequence that can be attached at its N- or C-terminus directly or indirectly to any of the FGF1 mutants provided herein to generate a chimeric protein. In addition, it can be linked to any of SEQ ID NOS: 48-49 via a linker and then the resulting chimera attached at its N- or C-terminus directly or indirectly to any of the FGF1 mutants provided herein to generate a chimeric protein.

SEQ ID NO: 48 provides an exemplary β-Klotho binding protein sequence that can be attached at its N- or C-terminus directly or indirectly to any of the FGF1 mutants provided herein to generate a chimeric protein. In addition, it can be linked to SEQ ID NO: 47 via a linker and then the resulting chimera attached at its N- or C-terminus directly or indirectly to any of the FGF1 mutants provided herein to generate a chimeric protein.

SEQ ID NO: 49 provides an exemplary β-Klotho binding protein sequence that can be attached at its N- or C-terminus directly or indirectly to any of the FGF1 mutants provided herein to generate a chimeric protein. In addition, it can be linked to SEQ ID NO: 47 via a linker and then the resulting chimera attached at its N- or C-terminus directly or indirectly to any of the FGF1 mutants provided herein to generate a chimeric protein.

SEQ ID NO: 50 provides an exemplary β-Klotho binding protein sequence that can be attached at its N- or C-terminus directly or indirectly to any of the FGF1 mutants provided herein to generate a chimeric protein.

SEQ ID NO: 51 provides an exemplary β-Klotho binding protein sequence that can be attached at its N- or C-terminus directly or indirectly to any of the FGF1 mutants provided herein to generate a chimeric protein.

SEQ ID NO: 52 provides an exemplary β-Klotho binding protein sequence that can be attached at its N- or C-terminus directly or indirectly to any of the FGF1 mutants provided herein to generate a chimeric protein.

SEQ ID NO: 53 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 54 (C2987) provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 55 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 56 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 57 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 58 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 59 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 60 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 61 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 62 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 63 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 64 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 65 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 66 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 67 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 68 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 69 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 70 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 71 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 72 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 73 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 74 provides an exemplary β-Klotho-FGFR1c binding protein sequence that can be attached at its N- or C-terminus directly or indirectly to any of the FGF1 mutants provided herein to generate a chimeric protein.

SEQ ID NO: 75 provides an exemplary β-Klotho-FGFR1c binding protein sequence that can be attached at its N- or C-terminus directly or indirectly to any of the FGF1 mutants provided herein to generate a chimeric protein.

SEQ ID NO: 76 provides an exemplary β-Klotho-FGFR1c binding protein sequence that can be attached at its N- or C-terminus directly or indirectly to any of the FGF1 mutants provided herein to generate a chimeric protein.

SEQ ID NO: 77 provides an exemplary β-Klotho-FGFR1c binding protein sequence that can be attached at its N- or C-terminus directly or indirectly to any of the FGF1 mutants provided herein to generate a chimeric protein.

SEQ ID NO: 78 provides an exemplary FGFR1c dimer chimera sequence (C2987).

SEQ ID NO: 79 provides an exemplary gorilla FGF1 protein sequence.

SEQ ID NO: 80 provides an exemplary chimpanzee FGF1 protein sequence.

SEQ ID NO: 81 provides an exemplary dog FGF1 protein sequence.

SEQ ID NO: 82 provides an exemplary cat FGF1 protein sequence.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Apr. 20, 2015. All references and GenBank® Accession numbers cited herein are incorporated by reference.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Administration: To provide or give a subject an agent, such as a mutated FGF1 protein disclosed herein, by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes.

Beta-Klotho binding domain or protein: A peptide sequence that binds selectively to β-Klotho (such as human β-Klotho, OMIM 61135, GenBank® Accession No. NP_783864.1), but not to other proteins. β-Klotho is a cofactor for FGF21 activity. Such a binding domain can include one or more monomers (wherein the monomers can be the same or different β-Klotho binding proteins), thereby generating a multimer (such as a dimer). In specific examples, such a domain/protein is not an antibody. Exemplary β-Klotho binding proteins can be found in SEQ ID NOS: 27, 28, 29, 30, 31, 32, 33, 34, 35, 36, 37, 38, 39, 40, 41, 42, 43, 44, 45, 46, 47, 48, 49, 50, 51, 52 and portions of SEQ ID NOS: 74, 75, 76, and 77, as well as U.S. Pat. No. 8,372,952, U.S. Publication No. 2013/0197191, and Smith et al., PLoS One 8:e61432, 2013, all herein incorporated by reference. Such β-Klotho binding proteins can be attached to the N-terminus, C-terminus, or both (e.g., directly or via linker), to any mutant FGF1 protein provided herein (e.g., any of SEQ ID NOS: 11, 12, 13, 14, 15, 16, 17, 18, 19, 20, 21, 22, 23, and 24).

A β-Klotho binding protein “specifically binds” to β-Klotho when the dissociation constant (K_{D}) is at least about 1×10⁻⁷ M, at least about 1.5×10⁻⁷, at least about 2×10⁻⁷, at least about 2.5×10⁻⁷, at least about 3×10⁻⁷, at least about at least about 5×10⁻⁷ M, at least about 1×10⁻⁸ M, at least about 5×10⁻⁸, at least about 1×10⁻⁹, at least about 5×10⁻⁹, at least about 1×10⁻¹⁰, or at least about 5×10⁻¹⁰ M. In one embodiment, K_{D} is measured by a radiolabeled antigen binding assay (RIA) performed with the β-Klotho binding protein and β-Klotho. In another example, K_{D} is measured using an ELISA assay.

C-terminal portion: A region of a protein sequence that includes a contiguous stretch of amino acids that begins at or near the C-terminal residue of the protein. A C-terminal portion of the protein can be defined by a contiguous stretch of amino acids (e.g., a number of amino acid residues).

Chimeric protein: A protein that includes at least a portion of the sequence of a full-length first protein (e.g., mutant FGF1 containing an S116 mutation) and at least a portion of the sequence of a full-length second protein (e.g., FGF19, FGF21, β-Klotho-binding protein, or FGF1Rc-binding protein), where the first and second proteins are different. A chimeric polypeptide also encompasses polypeptides that include two or more non-contiguous portions derived from the same polypeptide. The two different peptides can be joined directly or indirectly, for example using a linker.

Diabetes mellitus: A group of metabolic diseases in which a subject has high blood sugar, either because the pancreas does not produce enough insulin, or because cells do not respond to the insulin that is produced. Type 1 diabetes results from the body's failure to produce insulin. This form has also been called “insulin-dependent diabetes mellitus” (IDDM) or “juvenile diabetes.” Type 2 diabetes results from insulin resistance, a condition in which cells fail to use insulin properly, sometimes combined with an absolute insulin deficiency. This form is also called “non insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and in some examples diagnosed by demonstrating any one of:
a. Fasting plasma glucose level ≧7.0 mmol/l (126 mg/dl);
b. Plasma glucose ≧11.1 mmol/l (200 mg/dL) two hours after a 75 g oral glucose load as in a glucose tolerance test;
c. Symptoms of hyperglycemia and casual plasma glucose ≧11.1 mmol/l (200 mg/dl);
d. Glycated hemoglobin (Hb A1C) ≧6.5%

Effective amount or therapeutically effective amount: The amount of agent, such as a mutated FGF1 protein (or nucleic acid encoding such) disclosed herein, that is an amount sufficient to prevent, treat (including prophylaxis), reduce, and/or ameliorate the symptoms and/or underlying causes of any of a disorder or disease. In one embodiment, an “effective amount” is sufficient to reduce or eliminate a symptom of a disease, such as a diabetes (such as type II diabetes), for example by lowering blood glucose.

Fibroblast Growth Factor 1 (FGF1): e.g., OMIM 13220. Includes FGF1 nucleic acid molecules and proteins. FGF1 is a protein that binds to the FGF receptor and is also known as the acidic FGF. FGF1 sequences are publically available, for example from GenBank® sequence database (e.g., Accession Nos. NP_00791 and NP_034327 provide exemplary FGF1 protein sequences, while Accession Nos. NM_000800 and NM_010197 provide exemplary FGF1 nucleic acid sequences). One of ordinary skill in the art can identify additional FGF1 nucleic acid and protein sequences, including FGF1 variants.

Specific examples of native FGF1 sequences are provided in SEQ ID NOS: 1-5. A native FGF1 sequence is one that does not include a mutation that alters the normal activity of the protein (e.g., activity of SEQ ID NOS: 2, 4, or 5). A mature FGF1 sequence refers to an FGF1 peptide or protein product and/or sequence following any post-translational modification(s). A mutated FGF1 is a variant of FGF1 with different or altered biological activity, such as reduced mitogenicity (e.g., a variant of any of SEQ ID NOS: 1-5, such as one having at least 80%, at least 90%, at least 95%, at least 96%, at least 97%, at least 98% or at least 99% sequence identity to any of SEQ ID NOS: 1-5, but is not a native/wild-type sequence). In one example, such a variant includes a mutation at S116, for example in combination with an N-terminal truncation and/or one or more additional point mutations (such as one or more of those shown in Table 1), such as changes that decrease mitogenicity of FGF1, alter the heparin binding affinity of FGF1, and/or the thermostability of FGF1. Specific exemplary FGF1 mutant proteins are shown in SEQ ID NOS: 11, 12, 13, 14, 15, 16, 17, 18, 19, 20, 21, 22, 23, and 24. Mutated FGF1 proteins including a mutation at S116 can also be a chimera (e.g., include a portion of an FGF19 sequence, a portion of an FGF21 sequence, β-Klotho binding protein, FGFR1c binding protein, or combinations thereof).

Fibroblast Growth Factor 19 (FGF19): e.g., OMIM 603891. Includes FGF19 nucleic acid molecules and proteins. FGF19 regulates bile acid synthesis and has effects on glucose and lipid metabolism. FGF19 sequences are publically available, for example from the GenBank® sequence database (e.g., Accession Nos. NP_005108.1 and AAQ88669.1 provide exemplary FGF19 protein sequences, while Accession Nos. AY358302.1 and NM 005117.2 provide exemplary FGF19 nucleic acid sequences). One of ordinary skill in the art can identify additional FGF19 nucleic acid and protein sequences, including FGF19 variants.

Fibroblast Growth Factor 21 (FGF21): e.g., OMIM 609436. Includes FGF21 nucleic acid molecules and proteins. FGF21 stimulates glucose updated in adipocytes. FGF21 sequences are publically available, for example from the GenBank® sequence database (e.g., Accession Nos. AAQ89444.1, NP_061986, and AAH49592.1 provide exemplary FGF21 protein sequences, while Accession Nos. AY359086.1 and BC049592 provide exemplary FGF21 nucleic acid sequences). One of ordinary skill in the art can identify additional FGF21 nucleic acid and protein sequences, including FGF21 variants.

Fibroblast Growth Factor Receptor 1c (FGFR1c) binding domain or protein: A peptide sequence that binds selectively to FGFR1c (such as human FGFR1c, e.g., GenBank Accession No. NP_001167536.1 or NP_056934.2), but not to other proteins. FGFR1c is a component of the receptor complex mediating FGF21 activity. Such a binding domain can include one or more monomers (wherein the monomers can be the same or different sequences), thereby generating a multimer (such as a dimer). In specific examples, such a domain/protein is not an antibody. Exemplary FGFR1c-binding proteins can be found in SEQ ID NOS: 53, 54, 55, 56, 57, 58, 59, 60, 61, 62, 63, 64, 65, 66, 67, 68, 69, 70, 71, 72, 73 and portions of SEQ ID NOS: 74, 75, 76, and 77 or a multimer thereof such as SEQ ID NO: 78, as well as U.S. Pat. No. 8,372,952, U.S. Publication No. 2013/0197191, and Smith et al., PLoS One 8:e61432, 2013, all herein incorporated by reference. Thus, reference to a FGFR1c-binding protein multimer, includes proteins made using two or more peptides having at least 80%, at least 90%, at least 92%, at least 95%, at least 96%, at least 97%, at least 98%, at least 99% or 100% sequence identity to one or more of SEQ ID NO: 53, 54, 55, 56, 57, 58, 59, 60, 61, 62, 63, 64, 65, 66, 67, 68, 69, 70, 71, 72, 73, and 78. Such FGFR1c binding proteins (or an FGFR1c binding protein multimer) can be attached to the N-terminus, C-terminus, or both (e.g., directly or via linker), to any mutant FGF1 protein provided herein (e.g., any of SEQ ID NOS: 11, 12, 13, 14, 15, 16, 17, 18, 19, 20, 21, 22, 23 and 24).

A FGFR1c binding protein “specifically binds” to FGFR1c when the dissociation constant (K_{D}) is at least about 1×10⁻⁷ M, at least about 1.5×10⁻⁷, at least about 2×10⁻⁷, at least about 2.5×10⁻⁷, at least about 3×10⁻⁷, at least about at least about 5×10⁻⁷ M, at least about 1×10⁻⁸ M, at least about 5×10⁻⁸, at least about 1×10⁻⁹, at least about 5×10⁻⁹, at least about 1×10⁻¹⁰, or at least about 5×10⁻¹⁰ M. In one embodiment, K_{D} is measured by a radiolabeled antigen binding assay (RIA) performed with the FGFR1c-binding protein and FGFR1c. In another example, K_{D} is measured using an ELISA assay.

Fibroblast Growth Factor Receptor 1c (FGFR1c): Also known as FGFR1 isoform 2. Includes FGFR1c nucleic acid molecules and proteins. FGFR1c and β-Klotho can associate with FGF21 to form a signaling complex. FGFR1c sequences are publically available, for example from the GenBank® sequence database (e.g., Accession Nos. NP_001167536.1 and NP_056934.2 provide exemplary FGFR1c protein sequences). One of ordinary skill in the art can identify additional FGFR1c nucleic acid and protein sequences, including FGFR1c variants.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. Thus, host cells can be transgenic, in that they include nucleic acid molecules that have been introduced into the cell, such as a nucleic acid molecule encoding a mutant FGF1 protein disclosed herein.

Isolated: An “isolated” biological component (such as a mutated FGF1 protein or nucleic acid molecule) has been substantially separated, produced apart from, or purified away from other biological components in the cell of the organism in which the component naturally occurs, such as other chromosomal and extrachromosomal DNA and RNA, and proteins. Nucleic acids molecules and proteins which have been “isolated” thus include nucleic acids and proteins purified by standard purification methods. The term also embraces nucleic acid molecules and proteins prepared by recombinant expression in a host cell as well as chemically synthesized nucleic acids. A purified or isolated cell, protein, or nucleic acid molecule can be at least 70%, at least 80%, at least 90%, at least 95%, at least 96%, at least 97%, at least 98%, or at least 99% pure.

Linker: A moiety or group of moieties that joins or connects two or more discrete separate peptide or proteins, such as monomer domains, for example to generate a chimeric protein. In one example a linker is a substantially linear moiety. Exemplary linkers that can be used to generate the chimeric proteins provided herein include, but are not limited to: peptides, nucleic acid molecules, peptide nucleic acids, and optionally substituted alkylene moieties that have one or more oxygen atoms incorporated in the carbon backbone. A linker can be a portion of a native sequence, a variant thereof, or a synthetic sequence. Linkers can include naturally occurring amino acids, non-naturally occurring amino acids, or a combination of both. In one example a linker is composed of at least 5, at least 10, at least 15 or at least 20 amino acids, such as 5 to 10, 5 to 20, or 5 to 50 amino acids. In one example, the linker is a poly alanine.

Mammal: This term includes both human and non-human mammals. Similarly, the term “subject” includes both human and veterinary subjects (such as cats, dogs, cows, and pigs) and rodents (such as mice and rats).

Metabolic disorder/disease: A disease or disorder that results from the disruption of the normal mammalian process of metabolism. For example, a metabolic disorder/disease includes metabolic syndrome.

Other examples include, but are not limited to, (1) glucose utilization disorders and the sequelae associated therewith, including diabetes mellitus (Type I and Type-2), gestational diabetes, hyperglycemia, insulin resistance, abnormal glucose metabolism, “pre-diabetes” (Impaired Fasting Glucose (IFG) or Impaired Glucose Tolerance (IGT)), and other physiological disorders associated with, or that result from, the hyperglycemic condition, including, for example, histopathological changes such as pancreatic β-cell destruction; (2) dyslipidemias and their sequelae such as, for example, atherosclerosis, coronary artery disease, cerebrovascular disorders and the like; (3) other conditions which may be associated with the metabolic syndrome, such as obesity and elevated body mass (including the co-morbid conditions thereof such as, but not limited to, nonalcoholic fatty liver disease (NAFLD), nonalcoholic steatohepatitis (NASH), and polycystic ovarian syndrome (PCOS)), and also include thromboses, hypercoagulable and prothrombotic states (arterial and venous), hypertension, cardiovascular disease, stroke and heart failure; (4) disorders or conditions in which inflammatory reactions are involved, including atherosclerosis, chronic inflammatory bowel diseases (e.g., Crohn's disease and ulcerative colitis), asthma, lupus erythematosus, arthritis, or other inflammatory rheumatic disorders; (5) disorders of cell cycle or cell differentiation processes such as adipose cell tumors, lipomatous carcinomas including, for example, liposarcomas, solid tumors, and neoplasms; (6) neurodegenerative diseases and/or demyelinating disorders of the central and peripheral nervous systems and/or neurological diseases involving neuroinflammatory processes and/or other peripheral neuropathies, including Alzheimer's disease, multiple sclerosis, Parkinson's disease, progressive multifocal leukoencephalopathy, and Guillain-Barre syndrome; (7) skin and dermatological disorders and/or disorders of wound healing processes, including erythemato-squamous dermatoses; and (8) other disorders such as syndrome X, osteoarthritis, and acute respiratory distress syndrome. Other examples are provided in WO 2014/085365 (herein incorporated by reference).

In specific examples, the metabolic disease includes one or more of (such as at least 2 or at least 3 of): diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), and cardiovascular diseases (e.g., hypertension).

N-terminal portion: A region of a protein sequence that includes a contiguous stretch of amino acids that begins at or near the N-terminal residue of the protein. An N-terminal portion of the protein can be defined by a contiguous stretch of amino acids (e.g., a number of amino acid residues).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as a mutated FGF1 coding sequence). Generally, operably linked DNA sequences are contiguous and, where necessary to join two protein coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the disclosed mutated FGF1 proteins (or nucleic acid molecules encoding such) herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Recombinant: A recombinant nucleic acid molecule is one that has a sequence that is not naturally occurring (e.g., a mutated FGF1 protein) or has a sequence that is made by an artificial combination of two otherwise separated segments of sequence. This artificial combination can be accomplished by routine methods, such as chemical synthesis or by the artificial manipulation of isolated segments of nucleic acids, such as by genetic engineering techniques. Similarly, a recombinant protein is one encoded for by a recombinant nucleic acid molecule. Similarly, a recombinant or transgenic cell is one that contains a recombinant nucleic acid molecule and expresses a recombinant protein.

Sequence identity of amino acid sequences: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn, and tblastx. A description of how to determine sequence identity using this program is available on the NCBI website on the internet.

Variants of the mutated FGF1 proteins and coding sequences disclosed herein are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, a mutant FGF1 protein disclosed herein having a mutation at S116 (such as S116R), can share at least 80%, at least 85%, at least 90%, at least 91%, at least 92%, at least 95%, at least 96%, at least 97%, at least 98% or at least 99% sequence identity to SEQ ID NO: 5, but is not SEQ ID NO: 5 (which, in some examples, has one or more, such as 1, 2, 3, 4, 5, 6, 7, 8, 9, or 10 of the mutations or truncations shown in Tables 1 and 2). In addition, exemplary mutated FGF1 proteins having a mutation at S116 (such as S116R) have at least 80%, at least 85%, at least 90%, at least 92%, at least 95%, at least 96%, at least 97%, at least 98% or at least 99% sequence identity to SEQ ID NOS: 11, 13, 14, 15, 16, 17, 18, 19, 20, 21, 22, 23 or 24 and retain the ability to reduce blood glucose levels in vivo.

Similarly, exemplary mutated FGF1 coding sequences in some examples have at least 70%, at least 80%, at least 85%, at least 90%, at least 92%, at least 95%, at least 98%, or at least 99% sequence identity to SEQ ID NO: 10, as long as the coding sequence encodes a mutation at S116 that increases heparin binding affinity.

Similarly, exemplary β-Klotho-binding domain sequences that can be used in the mutant FGF1 chimeras disclosed herein in some examples have at least 70%, at least 80%, at least 85%, at least 90%, at least 92%, at least 95%, at least 97%, at least 98%, or at least 99% sequence identity to SEQ ID NO: 27, 28, 29, 30, 31, 32, 33, 34, 35, 36, 37, 38, 39, 40, 41, 42, 43, 44, 45, 46, 47, 48, 49, 50, 51, 52 or β-Klotho-binding portions of SEQ ID NOS: 74, 75, 76, and 77.

Similarly, exemplary FGFR1c binding sequences that can be used in the mutant FGF1 chimeras disclosed herein in some examples have at least 70%, at least 80%, at least 85%, at least 90%, at least 92%, at least 95%, at least 97%, at least 98%, or at least 99% sequence identity to SEQ ID NO: 53, 54, 55, 56, 57, 58, 59, 60, 61, 62, 63, 64, 65, 66, 67, 68, 69, 70, 71, 72, 73, or FGFR1c-binding portions of SEQ ID NOS: 74, 75, 76, and 77, or multimers such as SEQ ID NO: 78.

Subject: Any mammal, such as humans, non-human primates, pigs, sheep, cows, dogs, cats, rodents and the like which is to be the recipient of the particular treatment, such as treatment with a mutated FGF1 protein (or corresponding nucleic acid molecule) provided herein. In two non-limiting examples, a subject is a human subject or a murine subject. In some examples, the subject has one or more metabolic diseases, such as diabetes (e.g., type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), cardiovascular disease (e.g., hypertension), or combinations thereof. In some examples, the subject has elevated blood glucose.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses (Wolff, J. A., ed., Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA.

Transgene: An exogenous gene supplied by a vector. In one example, a transgene includes a mutated FGF1 coding sequence.

Vector: A nucleic acid molecule as introduced into a host cell, thereby producing a transformed host cell. A vector may include nucleic acid sequences that permit it to replicate in the host cell, such as an origin of replication. A vector may also include one or more mutated FGF1 coding sequences and/or selectable marker genes and other genetic elements known in the art. A vector can transduce, transform, or infect a cell, thereby causing the cell to express nucleic acids and/or proteins other than those native to the cell. A vector optionally includes materials to aid in achieving entry of the nucleic acid into the cell, such as a viral particle, liposome, protein coating, or the like.

### Overview

Provided herein are mutated FGF1 proteins that can include one or more mutations that increase its binding affinity for heparin and/or heparan sulfate, such as a mutation at S116. Such mutated FGF1 proteins can further include an N-terminal deletion, one or more additional point mutations (such as amino acid substitutions, deletions, additions, or combinations thereof), or combinations of an N-terminal deletion and additional one or more point mutations.

Also provided are methods of using the disclosed FGF1 mutant proteins (or their nucleic acid coding sequences) to lower glucose, for example to treat one or more metabolic diseases, or combinations thereof. Exemplary metabolic diseases that can be treated with the disclosed methods include, but are not limited to: type 2 diabetes, non-type 2 diabetes, type 1 diabetes, polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), cardiovascular diseases (e.g., hypertension), latent autoimmune diabetes (LAD), or maturity onset diabetes of the young (MODY).

In some examples, the FGF1 mutants containing an S116 mutation includes additional mutations that reduce its mitogenicity (e.g., relative to the mature wild-type FGF1, e.g., SEQ ID NO: 5), such as a reduction of at least 20%, at least 50%, at least 75% or at least 90%. For example, mutated FGF1 can be mutated to alter binding affinity for heparin and/or heparan sulfate compared to an FGF1 protein without the modification (e.g., a native or wild-type FGF1 protein). Methods of measuring mitogenicity are known in the art.

In some examples, the mutant FGF1 protein containing an S116 mutation is a truncated version of the mature protein (e.g., SEQ ID NO: 5), which can include for example deletion of at least 5, at least 6, at least 9, at least 10, at least 11, at least 12, at least 13, at least 14, at least 15, or at least 20 consecutive N-terminal amino acids, such as the N-terminal 5 to 10, 5 to 13, 5, 6, 7, 8, 9, 10, 11, 12, or 13 amino acids of mature FGF1. In some examples, such an N-terminally deleted FGF1 protein containing an S116 mutation includes has reduced mitogenic activity as compared to wild-type mature FGF1 protein. Specific examples of N-terminally deleted FGF1 proteins are shown in SEQ ID NOS: 6-9. Thus, any of SEQ ID NOS: 6-9 can be modified to include an S116 mutation (such as S116R).

In some examples, a mutated FGF1 containing an S116 mutation further includes one or more mutations that increase the thermostability (e.g., relative to mature or truncated FGF1, e.g., SEQ ID NO: 5), such as an increase of at least 20%, at least 50%, at least 75% or at least 90% compared to native FGF1. Exemplary mutations that can be used to increase the thermostability a mutated FGF1 containing an S116 mutation include, but are not limited to (a) one or more of K12V, C117V, C117P, C117T, C117S, C117A, (b) one or more of P134V, L44F, C83T, C83S, C83A C83V, C117V, C117P, C117T, C117S, C117A and F132W, and (c) one or more of L44F, M67I, L73V, V109L, L111I, C117V, C117P, C117T, C117S, C117A A103G, R119G, R119V, Δ104-106, and Δ120-122, wherein the numbering refers to SEQ ID NO: 5 (e.g., see Xia et al., PLoS One. 7:e48210, 2012). For example, mutated FGF1 containing an S116 mutation can be mutated to increase the thermostability of the protein relative to an FGF1 protein without the modification. Methods of measuring thermostability are known in the art. In one example, the method provided in Xia et al., PLoS One. 7:e48210, 2012 is used.

In some examples, the mutant FGF1 protein containing an S116 mutation includes one or more additional mutations, such as at least 1, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 amino acid substitutions, such as 1-20, 1-10, 4-8, 5-25, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 amino acid substitutions (such as those shown in Table 1). In some examples, the mutant FGF1 protein containing an S116 mutation further includes deletion of one or more amino acids, such as deletion of 1-10, 4-8, 5-10, 1, 2, 3, 4, 5, 6, 7, 8, 9, 10, 11, 12, 13, 14, 15, 16, 17, 18, 19, or 20 amino acid deletions. In some examples, the mutant FGF1 protein containing an S116 mutation further includes a combination of amino acid substitutions and deletions, such as at least 1 substitution and at least 1 deletion, such as 1 to 10 substitutions with 1 to 10 deletions.

Exemplary mutations that can be made to a mutant FGF1 protein containing an S116 mutation (such as S116R) are shown in Table 1 below, with amino acids referenced to either SEQ ID NOS: 2 or 5. One skilled in the art will recognize that these mutations can be used singly, or in any combination (such as 1-20, 1-10, 4-8, 5-25, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 of these amino acid substitutions and/or deletions). In addition, such mutant FGF1 proteins can be part of a chimeric protein, such as with FGF19, FGF21, a protein that selectively binds to β-Klotho, or a protein that selectively binds to FGFR1c.

TABLE 1
Exemplary FGF1 mutations

| Location of | | |
|---|---|---|
| Point Mutation | | Location of Point |
| Position in | | Mutation Position in SEQ |
| SEQ ID NO: 2 | Mutation Citation | ID NO: 5 |
| K24 | K9T | K9 |
| K25 | K10T | K10 |
| K27 | K12V | K12 |
| L29 | L14A | L14 |
| Y30 | Y15F, Y15A, Y15V | Y15 |
| C31 | C16V, C16A, C16T, C16S | C16 |
| H36 | H21Y | H21 |
| R50 | R35E, R35V | R35 |
| Q55 | Q40P | Q40 |
| L59 | L44F | L44 |
| L61 | L46V | L46 |
| S62 | S47I | S47 |
| E64 | E49Q, E49A | E49 |
| Y70 | Y55F, Y55S, Y55A, Y55W | Y55 |
| M82 | M67I | M67 |
| L88 | L73V | L73 |
| C98 | C83T, C83S, C83A C83V | C83 |
| E102 | E87V, E87A, E87S, E87T, | E87 |
| | E87H | |
| H108 | H93G, H93A | H93 |
| Y109 | Y94V, Y94F, Y94A | Y94 |
| N110 | N95V, N95A, N95S, N95T | N95 |
| H117 | H102Y | H102 |
| A118 | A103G | A103 |
| EKN 119-121 | Δ104-106 | EKN (104-106) |
| F123 | F108Y | F108 |
| V124 | V109L | V109 |
| L126 | L111I | L111 |
| K127 | K112D, K112E, K112Q | K112 |
| K128 | K113Q, K113E, K113D | K113 |
| C132 | C117V, C117P, C117T, | C117 |
| | C117S, C117A | |
| K133 | K118N, K118E, K118V | K118 |
| R134 | R119G, R119V, R119E | R119 |
| GPR 135-137 | Δ120-122 | GPR (120-122) |
| F147 | F132W | F132 |
| L148 | L133A, L133S | L133 |
| P149 | P134V | P134 |
| L150 | L135A, L135S | L135 |

In some examples, the mutant FGF1 protein containing an S116 mutation (such as S116R) further includes mutations at one or more of the following positions: K9, K10, K12, L14, Y15, C16, H21, R35, Q40, L44, L46, S47, E49, Y55, M67, L73, C83, L86, E87, H93, Y94, N95, H102, A103, E104, K105, N106, F108, V109, L111, K112, K113, C117, K118, R119, G120, P121, R122, F132, L133, P134, L135, such as one or more of K9, K10, K12, K112, K113, such as 1 to 5, 2 to 5, 3 to 6, 3 to 8, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or all of these positions. In one example, K9 and K10 are replaced with DQ.

In some examples, the mutant FGF1 protein containing an S116 mutation (such as S116R) further includes mutations at 1, 2, 3, 4, 5, 6, 7, or 8 of the following positions: K12, R35, E49, Y55, E87, Y94, N95, and C117 (wherein the numbering refers to SEQ ID NO: 5), such as one or more of K12V, R35E, R35V, E49Q, E49A, Y55F, Y55S, Y55A, Y55W, E87V, E87A, E87S, E87T, E87H, Y94V, Y94F, Y94A, N95V, N95A, N95S, N95T, C117V, C117P, C117T, C117S, and C117A (such as 1, 2, 3, 4, 5, 6, 7, or 8 of these mutations). For example, E87 or N95 can be replaced with a non-charged amino acid. In addition, Y15 and/or Y94 can be replaced with an amino acid that destabilizes the hydrophobic interactions.

In some examples, the mutant FGF1 protein containing an S116 mutation (such as S116R) further includes mutations on either side of Y15, E87, Y94, and N95, such as one or more of L14, C16, H93, and T96, such as mutations at 1, 2, 3, or 4 of these positions.

In some examples, the mutant FGF1 protein containing an S116 mutation (such as S116R) further includes mutations at 1, 2, 3, 4, 5, or 6 of the following positions: Y15, C16, E87, H93, Y94, and N95 (wherein the numbering refers to SEQ ID NO: 5), such as one or more of Y15F, Y15A, Y15V, E87V, E87A, E87S, E87T, E87H, H93A, N95V, N95A, N95S, N95T, Y94V, Y94F, and Y94A (such as 1, 2, 3, 4, 5, or 6 of these mutations).

In some examples, the mutant FGF1 protein containing an S116 mutation (such as S116R) further includes mutations at one or more of the following positions: C16, C83, and C117 (wherein the numbering refers to SEQ ID NO: 5), such as one or more of C16V, C16A, C16T, C16S, C83T, C83S, C83A C83V, C117V, C117P, C117T, C117S, and C117A (such as 1, 2, or 3 of these mutations).

In some examples, the mutant FGF1 protein containing an S116 mutation (such as S116R) further includes mutations at only one or two of the following positions: E87, Y94, and N95 (wherein the numbering refers to SEQ ID NO: 5), such as one or two of E87V, E87A, E87S, E87T, E87H, Y94V, Y94F, Y94A, N95V, N95A, N95S, and N95T.

In some examples, the mutant FGF1 protein containing an S116 mutation (such as S116R) further includes mutations at 1, 2, or 3 of the following positions: K12, N95, and C117 (wherein the numbering refers to SEQ ID NO: 5), such as one or more of K12V, K12C, N95V, N95A, N95S, N95T, C117V, C117P, C117T, C117S, and C117A (such as 1, 2, or 3 of these mutations, such as K12V, C83T, and C117V).

In some examples, the mutant FGF1 protein containing an S116 mutation (such as S116R) further includes mutations at residues that interact with the FGF1 receptor, such as Y15, E87, Y94, and N95. Thus, in some examples, 1, 2, 3, or 4 of these positions are further mutated, for example the amino acid at position 87 and/or 95 of SEQ ID NO: 5 can be changed to a V, A, S or T. In some examples, the amino acid at position 15 and/or 95 of SEQ ID NO: 5 can be changed to a V, A, or F. In some examples, combinations of these changes are made.

In some examples, the mutant FGF1 protein containing an S116 mutation (such as S116R) further includes a mutation at K12 of FGF1, which is predicted to be at the receptor interface. Thus, K12 of SEQ ID NO: 5 can be mutated, for example to a V or C.

In some examples, the mutant FGF1 protein containing an S116 mutation (such as S116R) further includes mutations at the heparin binding site. For example, amino acids K112, K113, and K118 can be mutated, for example to a E, Q, N, V or D, such as a N, E or V at position K118, and a D, E or Q at positions K112 and K113.

In some examples, the mutant FGF1 protein containing an S116 mutation (such as S116R) further includes a mutation at R35 of SEQ ID NO: 5, which forms a salt bridge with the D2 domain of the FGF receptor, and thus can be mutated, for example to an E or V. In some examples, the mutant FGF1 protein containing an S116 mutation (such as S116R) further includes an R35E mutation (wherein the numbering refers to SEQ ID NO: 5).

In some examples, the mutant FGF1 protein containing an S116 mutation (such as S116R) further includes one or more of K12V, L46V, R35E, R35V, E87V, E87H, N95V, N95T, K118N, K118E, C117V, and P134V (wherein the numbering refers to SEQ ID NO: 5). In some examples, the point mutation includes replacing amino acid sequence ILFLPLPV (amino acids 145-152 of SEQ ID NOS: 2 and 4) to AAALPLPV, ILALPLPV, ILFAPLPV, or ILFLPAPA. In some examples, such an FGF1 protein with one or more point mutations has reduced mitogenic activity as compared to wild-type mature FGF1 protein.

In some examples, the mutant FGF1 protein containing an S116 mutation (such as S116R) includes at least 90 consecutive amino acids from amino acids 5-141 of FGF1 (e.g., of SEQ ID NOS: 2 or 4), (which in some examples can include further deletion of N-terminal amino acids 1-20 and/or point mutations, such as substitutions, deletions, or additions). In some examples, the mutant FGF1 protein containing an S116 mutation (such as S116R) includes at least 100 or at least 110 consecutive amino acids from amino acids 5-141 of FGF1, such as at least 100 consecutive amino acids from amino acids 5-141 of SEQ ID NOS: 2 or 4 or at least 100 consecutive amino acids from SEQ ID NO: 5.

In some examples, the mutant FGF1 protein containing an S116 mutation (such as S116R) further includes both an N-terminal truncation and additional point mutations. Specific exemplary FGF1 mutant proteins containing an S116 mutation (such as S116R) are shown in SEQ ID NOS: 11, 13, 14, 15, 16, 17, 18, 19, 20, 21, 22, 23, and 24. In some examples, the FGF1 mutant includes an N-terminal deletion, but retains a methionine at the N-terminal position. In some examples, the FGF1 mutant containing an S116 mutation (such as S116R) is 120-140 or 125-140 amino acids in length.

In some examples, the FGF1 mutant protein containing an S116 mutation (such as S116R) is part of a chimeric protein. For example, one end of the mutant FGF1 mutant protein can be joined directly or indirectly to the end of FGF19 or FGF21, such as a C-terminal region of FGF 19 or FGF21. In some examples, the mutated FGF1 portion of the chimera is at the N-terminus of the chimera, and the FGF19 or FGF21 portion is the C-terminus of the chimera. However, this can be reversed, such that the mutated FGF1 portion of the chimera is the C-terminus of the chimera, and the FGF19 or FGF21 portion is the N-terminus of the chimera. For example, at least 10, at least 20, at least 30, at least 40, at least 41, at least 42, at least 43, at least 44, at least 45, at least 46, at least 47, at least 48, at least 49, at least 50 or at least 60 C-terminal amino acids of FGF19 or FGF21 (such as the C-terminal 60, 55, 50, 49, 48, 47, 46, 45, 44, 43, 42, 41, 40, 35, 30, 25, 20, 15 or 10 amino acids) can be part of the chimera. Examples of C-terminal fragments of FGF21 and FGF19 that can be used are shown in SEQ ID NOS: 25 and 26, respectively. In some examples, the mutant FGF1 and FGF21 or FGF19 portion are linked indirectly through the use of a linker, such as one composed of at least 5, at least 10, at least 15 or at least 20 amino acids. In one example, the linker is a poly alanine.

In some examples, the FGF1 mutant protein containing an S116 mutation (such as S116R) is part of a chimeric protein with a β-Klotho-binding protein. For example, one end of the mutant FGF1 mutant protein can be joined directly or indirectly to the end of a β-Klotho-binding protein. In some examples, the mutated FGF1 portion of the chimera is at the N-terminus of the chimera, and the β-Klotho-binding protein portion is the C-terminus of the chimera. However, this can be reversed, such that the mutated FGF1 portion of the chimera is the C-terminus of the chimera, and the β-Klotho binding protein portion is the N-terminus of the chimera. Examples of β-Klotho-binding proteins that can be used are shown in SEQ ID NOS: 27, 28, 29, 30, 31, 32, 33, 34, 35, 36, 37, 38, 39, 40, 41, 42, 43, 44, 45, 46, 47, 48, 49, 50, 51, 52, and portions of SEQ ID NOS: 74, 75, 76, and 77. In some examples, the mutant FGF1 and β-Klotho-binding protein portion are linked indirectly through the use of a linker, such as one composed of at least 5, at least 10, at least 15 or at least 20 amino acids. In one example, the linker is a poly alanine.

In some examples, the FGF1 mutant protein containing an S116 mutation (such as S116R) is part of a chimeric protein with an FGFR1c-binding protein. For example, one end of the mutant FGF1 mutant protein can be joined directly or indirectly to the end of an FGFR1c-binding protein. In some examples, the mutated FGF1 portion of the chimera is at the N-terminus of the chimera, and the FGFR1c-binding protein portion is the C-terminus of the chimera. However, this can be reversed, such that the mutated FGF1 portion of the chimera is the C-terminus of the chimera, and the FGFR1c-binding protein portion is the N-terminus of the chimera. Examples of FGFR1c-binding proteins that can be used are shown in SEQ ID NOS: 53, 54, 55, 56, 57, 58, 59, 60, 61, 62, 63, 64, 65, 66, 67, 68, 69, 70, 71, 72, 73, and FGFR1c-binding portions of SEQ ID NOS: 74, 75, 76, and 77. In some examples, the mutant FGF1 and FGFR1c-binding protein portion are linked indirectly through the use of a linker, such as one composed of at least 5, at least 10, at least 15 or at least 20 amino acids. In one example, the linker is a poly alanine.

In some examples, the FGF1 mutant protein containing an S116 mutation (such as S116R) is part of a chimeric protein with both an FGFR1c-binding protein and a β-Klotho-binding protein, in any order. For example, one end of the mutant FGF1 mutant protein can be joined directly or indirectly to the end of an FGFR1c-binding/β-Klotho-binding or β-Klotho-binding/FGFR1c-binding chimeric protein. In some examples, the mutated FGF1 portion of the chimera is at the N-terminus of the chimera, and the FGFR1c-binding/β-Klotho-binding or β-Klotho-binding/FGFR1c-binding chimeric protein portion is the C-terminus of the chimera. However, this can be reversed, such that the mutated FGF1 portion of the chimera is the C-terminus of the chimera, and the FGFR1c-binding/β-Klotho-binding or β-Klotho-binding/FGFR1c-binding chimeric protein portion is the N-terminus of the chimera. In one example the FGFR1c-binding/β-Klotho-binding or β-Klotho-binding/FGFR1c-binding chimeric protein is any one of those shown in SEQ ID NOS: 74, 75, 76, and 77. In some examples, the mutant FGF1 and FGFR1c-binding/β-Klotho-binding or β-Klotho-binding/FGFR1c-binding chimeric protein portion are linked indirectly through the use of a linker, such as one composed of at least 5, at least 10, at least 15 or at least 20 amino acids. In one example, the linker is a poly alanine.

In some examples, the FGF1 mutant protein containing an S116 mutation (such as S116R) includes at least 80% sequence identity to SEQ ID NO: 11, 13, 14, 15, 16, 17, 18, 19, 20, 21, 22, 23, or 24. Thus, the FGF1 mutant protein containing an S116 mutation (such as S116R) can have at least 90%, at least 95%, at least 96%, at least 97%, at least 98% or at least 99% sequence identity to any of SEQ ID NOS: 11, 13, 14, 15, 16, 17, 18, 19, 20, 21, 22, 23, and 24 (but is not a native FGF1 sequence, such as SEQ ID NO: 5). In some examples, the FGF1 mutant protein containing an S116 mutation (such as S116R) includes or consists of SEQ ID NO: 11, 13, 14, 15, 16, 17, 18, 19, 20, 21, 22, 23 or 24. The disclosure encompasses variants of the disclosed FGF1 mutant proteins, containing an S116 mutation (such as S116R) such as SEQ ID NO: 11, 13, 14, 15, 16, 17, 18, 19, 20, 21, 22, 23, or 24, having 1 to 8, 2 to 10, 1 to 5, 1 to 6, or 5 to 10 additional mutations, such as conservative amino acid substitutions.

Also provided are isolated nucleic acid molecules encoding the disclosed mutated FGF1 proteins containing an S116 mutation (such as S116R), such as a nucleic acid molecule encoding a protein having at least 80%, at least 85%, at least 90%, at least 95%, at least 98%, or at least 99% sequence identity to SEQ ID NOS: 11, 13, 14, 15, 16, 17, 18, 19, 20, 21, 22, 23, or 24 (but is not a native FGF1 sequence). One exemplary coding sequence is shown in SEQ ID NO: 10; thus, the disclosure provides sequences having at least 80%, at least 85%, at least 90%, at least 95%, at least 98%, or at least 99% sequence identity to any of SEQ ID NO: 10. Vectors and cells that include such nucleic acid molecules are also provided. For example, such nucleic acid molecules can be expressed in a host cell, such as a bacterium or yeast cell (e.g., E. coli), thereby permitting expression of the mutated FGF1 protein containing an S116 mutation (such as S116R). The resulting mutated FGF1 protein containing an S116 mutation (such as S116R) can be purified from the cell.

Methods of using the disclosed mutated FGF1 proteins containing an S116 mutation (such as S116R) are provided. As discussed herein, the mutated mature FGF1 protein can include a deletion of at least six contiguous N-terminal amino acids, at least one additional point mutation, or combinations thereof. For example, such methods include administering a therapeutically effective amount of a disclosed mutated FGF1 protein containing an S116 mutation (such as S116R) (such as at least 0.01 mg/kg, at least 0.05 mg/kg, at least 0.1 mg/kg, at least 0.2 mg/kg, or at least 0.5 mg/kg) (or nucleic acid molecules encoding such) to reduce blood glucose in a mammal, such as a decrease of at least 5%, at least 10%, at least 25% or at least 50%, for example as compared to administration of no mutant FGF1 mutant protein containing an S116 mutation (such as S116R) (e.g., administration of PBS).

In one example, the method is a method of reducing fed and fasting blood glucose, improving insulin sensitivity and glucose tolerance, reducing systemic chronic inflammation, ameliorating hepatic steatosis in a mammal, reducing triglycerides, decreasing insulin resistance, reducing hyperinsulinemia, increasing glucose tolerance, reducing hyperglycemia, reducing food intake, or combinations thereof. Such a method can include administering a therapeutically effective amount of a disclosed mutated FGF1 protein containing an S116 mutation (such as S116R) (such as at least 0.01 mg/kg, at least 0.05 mg/kg, at least 0.1 mg/kg, at least 0.2 mg/kg, or at least 0.5 mg/kg) (or nucleic acid molecules encoding such) to reduce fed and fasting blood glucose, improve insulin sensitivity and glucose tolerance, reduce systemic chronic inflammation, ameliorate hepatic steatosis in a mammal, reduce food intake, or combinations thereof.

In one example, the method is a method of treating a metabolic disease (such as metabolic syndrome, diabetes, or obesity) in a mammal. Such a method can include administering a therapeutically effective amount of a disclosed mutated FGF1 protein containing an S116 mutation (such as S116R) (such as at least 0.01 mg/kg, at least 0.05 mg/kg, at least 0.1 mg/kg, at least 0.2 mg/kg, or at least 0.5 mg/kg) (or nucleic acid molecules encoding such) to treat the metabolic disease.

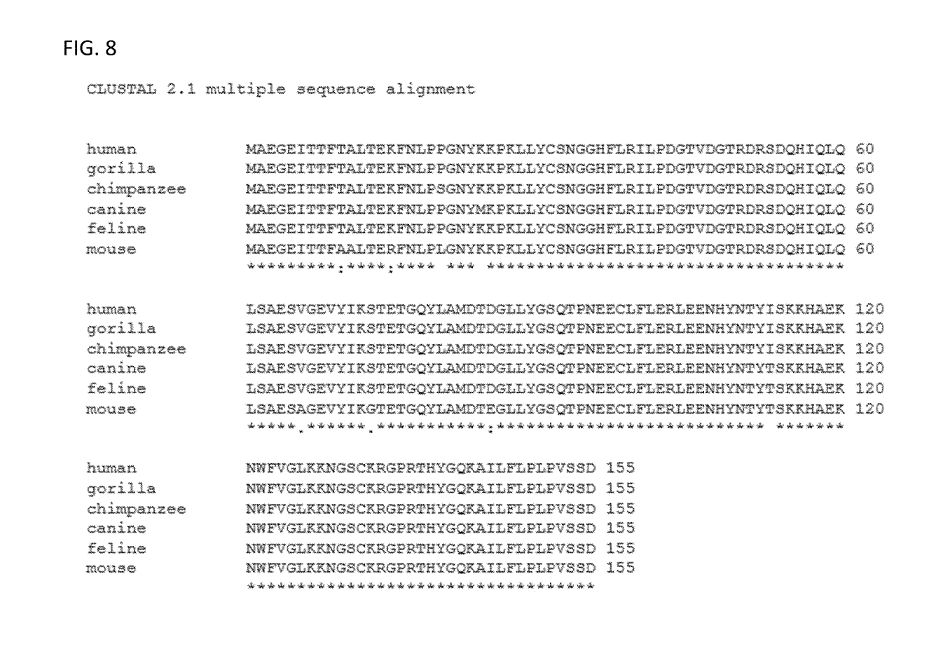

In some examples, the mammal, such as a human, cat, or dog, has diabetes. Methods of administration are routine, and can include subcutaneous, intraperitoneal, intramuscular, or intravenous injection or infusion. In some examples, the mutated FGF1 protein is a mutated canine FGF1 protein, and is used to treat a dog. For example, a canine FGF1 (such as XP_849274.1) can be mutated to include an S131 mutation (referring to amino acid 131 in XP_849274.1), such as S131R, which is analogous to the human S116R mutation. This mutation can also be used in combination with, for example, an N-terminal deletion, and/or one or more additional point mutations. Similarly, in some embodiments, the mutated FGF1 protein containing an S116 mutation (such as S116R) is a mutated cat FGF1 protein, and is used to treat a cat. Thus, for example, a feline FGF1 (such as XP_011281008.1) can be mutated to include an 5131 mutation (which is amino acid 131 in XP_011281008.1), such as S131R, and can also be used in combination with an N-terminal deletion and/or one or more additional point mutations. Based on routine methods of sequence alignment (e.g., see FIG. 8), one skilled in the art can mutate any known FGF1 sequence to generate mutations that correspond to those provided herein (for example, the FGF1 sequence can be selected based on the subject to be treated, e.g., a dog can be treated with a mutated canine FGF1 protein or corresponding nucleic acid molecule).

In some examples, use of the FGF1 mutants containing an S116 mutation (such as S116R disclosed herein does not lead to (or significantly reduces, such as a reduction of at least 20%, at least 50%, at least 75%, or at least 90%) the adverse side effects observed with thiazolidinediones (TZDs) therapeutic insulin sensitizers, including weight gain, increased liver steatosis and bone fractures (e.g., reduced effects on bone mineral density, trabecular bone architecture and cortical bone thickness).

Provided are methods of reducing fed and fasting blood glucose, improving insulin sensitivity and glucose tolerance, reducing systemic chronic inflammation, ameliorating hepatic steatosis, reducing food intake, or combinations thereof, in a mammal, such as within 12 hours, within 24 hours, or within 48 hours of the treatment, such as within 12 to 24 hours, within 12 to 36 hours, or within 24 to 48 hours. Such methods can include administering a therapeutically effective amount of a FGF1 mutant containing an S116 mutation (such as S116R) disclosed herein, to the mammal, or a nucleic acid molecule encoding the FGF1 mutant or a vector comprising the nucleic acid molecule, thereby reducing fed and fasting blood glucose, improving insulin sensitivity and glucose tolerance, reducing systemic chronic inflammation, ameliorating hepatic steatosis, reduce one or more non-HDL lipid levels, reduce food intake, or combinations thereof, in a mammal. In some examples, the fed and fasting blood glucose is reduced in the treated subject by at least 10%, at least 20%, at least 30%, at least 50%, at least 75%, or at least 90% as compared to an absence of administration of the FGF1 mutant. In some examples, insulin sensitivity and glucose tolerance is increased in the treated subject by at least 10%, at least 20%, at least 30%, at least 50%, at least 75%, or at least 90% as compared to an absence of administration of the FGF1 mutant. In some examples, systemic chronic inflammation is reduced in the treated subject by at least 10%, at least 20%, at least 30%, at least 50%, at least 75%, or at least 90% as compared to an absence of administration of the FGF1 mutant. In some examples, hepatic steatosis is reduced in the treated subject by at least 10%, at least 20%, at least 30%, at least 50%, at least 75%, or at least 90% as compared to an absence of administration of the FGF1 mutant. In some examples, one or more lipids (such as a non-HDL, for example IDL, LDL and/or VLDL) are reduced in the treated subject by at least 10%, at least 20%, at least 30%, at least 50%, at least 75%, or at least 90% as compared to an absence of administration of the FGF1 mutant In some examples, triglyceride and or cholesterol levels are reduced with the FGF1 mutant by at least 10%, at least 20%, at least 30%, at least 50%, at least 75%, or at least 90% as compared to an absence of administration of the FGF1 mutant. In some examples, the amount of food intake is reduced in the treated subject by at least 10%, at least 20%, at least 30%, at least 50%, at least 75%, or at least 90% as compared to an absence of administration of the FGF1 mutant (such as within 12 hours, within 24 hours, or within 48 hours of the treatment, such as within 12 to 24 hours, within 12 to 36 hours, or within 24 to 48 hours). In some examples, combinations of these reductions are achieved.

### Mutated FGF1 Proteins

The present disclosure provides mutated FGF1 proteins containing an S116 mutation (such as S116R), for example an S116 mutation that increases its binding affinity for heparin and/or heparan sulfate. In some examples, such mutants further include an N-terminal deletion, one or more point mutations (such as amino acid substitutions, deletions, additions, or combinations thereof), or combinations of N-terminal deletions and one or more additional point mutations. Such proteins and corresponding coding sequences can be used in the methods provided herein. In some examples, the disclosed FGF1 mutant proteins have reduced mitogenicity compared to mature native FGF1 (e.g., SEQ ID NO: 5), such as a reduction of at least 20%, at least 50%, at least 75% or at least 90%. For example, FGF1 can be mutated to alter (e.g., increase or decrease) binding affinity for heparin and/or heparan sulfate compared to a native FGF1 protein without the modification. Methods of measuring mitogenicity and heparin binding are known in the art.

In some examples, the mutant FGF1 protein containing an S116 mutation (such as S116R) is a truncated version of the mature protein (e.g., SEQ ID NO: 5), which can include for example deletion of at least 5, at least 6, at least 9, at least 10, at least 11, at least 12, at least 13, at least 14, at least 15, or at least 20 consecutive N-terminal amino acids. Thus, in some examples, the mutant FGF1 protein containing an S116 mutation (such as S116R) is a truncated version of the mature protein (e.g., SEQ ID NO: 5), such a deletion of the N-terminal 5, 6, 7, 8, 9, 10, 11, 12, 13, 14, 15, 16, 17, 18, 19 or 20 amino acids shown in SEQ ID NO: 5. Examples of N-terminally truncated FGF1 proteins are shown in SEQ ID NOS: 6, 7, 8, 9, 14, 19, 21, 22, 23, and 24. In some examples, the FGF1 mutant containing an S116 mutation (such as S116R) includes an N-terminal deletion, but retains a methionine at the N-terminal position. In some examples, such an N-terminally deleted FGF1 protein containing an S116 mutation (such as S116R) has reduced mitogenic activity as compared to wild-type mature FGF1 protein.

Thus, in some examples, the mutant FGF1 protein containing an S116 mutation (such as S116R) includes at least 90 consecutive amino acids from amino acids 5-141 or 5-155 of FGF1 (e.g., of SEQ ID NOS: 2 or 4), (which in some examples can include further deletion of N-terminal amino acids 1-20 and/or point mutations, such as substitutions, deletions, and/or additions). In some examples, the mutant FGF1 protein containing an S116 mutation (such as S116R) includes at least 90 consecutive amino acids from amino acids 1-140 of FGF1 (e.g., of SEQ ID NO: 5), (which in some examples can include further deletion of N-terminal amino acids 1-20 and/or point mutations, such as substitutions, deletions, and/or additions). Thus, in some examples, the mutant FGF1 protein containing an S116 mutation (such as S116R) includes at least 90 consecutive amino acids from amino acids 5-141 of FGF1, such as at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 115, at least 120, at least 125, or at least 130 consecutive amino acids from amino acids 5-141 of SEQ ID NOS: 2 or 4 (such as 90-115, 90-125, 90-100, or 90-95 consecutive amino acids from amino acids 5-141 of SEQ ID NOS: 2 or 4). In some examples, the mutant FGF1 protein containing an S116 mutation (such as S116R) includes least 90 consecutive amino acids from SEQ ID NO: 5. Thus, in some examples, the mutant FGF1 protein includes at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, or at least 110 consecutive amino acids from SEQ ID NO: 5 (such as 90-115, 90-100, or 90-95 consecutive amino acids from SEQ ID NO: 5).

In some examples, the mutant FGF1 protein containing an S116 mutation (such as S116R) further includes at least 1, at least 4, at least 5, at least 6, at least 7, at least 8, at least 9, at least 10, at least 11, at least 12, at least 13, at least 14, at least 15, at least 16, at least 17, at least 18, at least 19, or at least 20 additional amino acid substitutions, such as 1-20, 1-10, 4-8, 5-12, 5-10, 5-25, 1, 2, 3, 4, 5, 6, 7, 8, 9, 10, 11, 12, 13, 14, or 15 additional amino acid substitutions. For example, point mutations can be introduced into an FGF1 sequence containing an S116 mutation (such as S116R) to decrease mitogenicity, increase stability, alter binding affinity for heparin and/or heparan sulfate (compared to the portion of a native FGF1 protein without the modification), or combinations thereof. Specific exemplary point mutations that can be used are shown above in Table 1.

In some examples, the mutant FGF1 protein having a mutation at S116 (such as S116R) includes one or more additional mutations (such as a substitution or deletion) at one or more of the following positions K9, K10, K12, L14, Y15, C16, H21, R35, Q40, L44, L46, S47, E49, Y55, M67, L73, C83, L86, E87, H93, Y94, N95, H102, A103, E104, K105, N106, F108, V109, L111, K112, K113, C117, K118, R119, G120, P121, R122, F132, L133, P134, L135, such as one or more of K9, K10, K12, K112, K113, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or all 42 of these positions. In some examples the mutant FGF1 protein having a mutation at S116 (such as S116R) further has as one or more of K9T, K10T, K12V, L14A, Y15F, Y15A, Y15V, C16V, C16A, C16T, C16S, H21Y, R35E, R35V, Q40P, L44F, L46V, S47I, E49Q, E49A, Y55F, Y55S, Y55A, Y55W, M67I, L73V, C83T, C83S, C83A C83V, E87V, E87A, E87S, E87T, H93G, H93A, Y94V, Y94F, Y94A, N95V, N95A, N95S, N95T, H102Y, A103G, A104-106, F108Y, V109L, L111I, K112D, K112E, K112Q, K113Q, K113E, K113D, C117V, C117P, C117T, C117S, C117A, K118N, K118E, K118V, R119G, R119V, R119E, A120-122, F132W, L133A, L133S, P134V, L135A, L135S, (wherein the numbering refers to SEQ ID NO: 5), such as 1 to 5, 1 to 10, 2 to 5, 2 to 10, 2 to 20, 5 to 10, 5 to 40, or 5 to 20 of these mutations, such as 1, 2, 3, 4, 5, 6, 7, 8, 9, 10, 11, 12, 13, 14, 15, 16, 17, 18, 19 or 20 of these mutations.

In some examples, the mutant FGF1 protein containing an S116 mutation (such as S116R) further includes mutations at 1, 2, 3, 4, 5, 6, 7, or 8 of the following positions: K12, R35, E49, Y55, E87, Y94, N95, and C117 (wherein the numbering refers to SEQ ID NO: 5), such as one or more of K12V, R35E, R35V, E49Q, E49A, Y55F, Y55S, Y55A, Y55W, E87V, E87A, E87S, E87T, E87H, Y94V, Y94F, Y94A, N95V, N95A, N95S, N95T, C117V, C117P, C117T, C117S, and C117A (such as 1, 2, 3, 4, 5, 6, 7, or 8 of these mutations). For example, E87 or N95 can be replaced with a non-charged amino acid. In addition, Y15 and/or Y94 can be replaced with an amino acid that destabilizes the hydrophobic interactions.

In some examples, the mutant FGF1 protein containing an S116 mutation (such as S116R) further includes mutations at 1, 2, 3, 4, 5, or 6 of the following positions: Y15, C16, E87, H93, Y94, and N95 (wherein the numbering refers to SEQ ID NO: 5), such as one or more of Y15F, Y15A, Y15V, E87V, E87A, E87S, E87T, E87H, H93A, N95V, N95A, N95S, N95T, Y94V, Y94F, and Y94A (such as 1, 2, 3, 4, 5, or 6 of these mutations).

In some examples, the mutant FGF1 protein containing an S116 mutation (such as S116R) further includes mutations at only one or two of the following positions: E87, Y94, and N95 (wherein the numbering refers to SEQ ID NO: 5), such as one or two of E87V, E87A, E87S, E87T, E87H, Y94V, Y94F, Y94A, N95V, N95A, N95S, and N95T.

In some examples, the mutant FGF1 protein containing an S116 mutation (such as S116R) further includes mutations at 1, 2, or 3 of the following positions: K12, N95, and C117 (wherein the numbering refers to SEQ ID NO: 5), such as one or more of K12V, K12C, N95V, N95A, N95S, N95T, C117V, C117P, C117T, C117S, and C117A (such as 1, 2, or 3 of these mutations, such as K12V, C83T, and C117V).

In some examples, the mutant FGF1 protein containing an S116 mutation (such as S116R) further includes mutations at residues that interact with the FGF1 receptor, such as Y15, E87, Y94, and N95. Thus, in some examples, 1, 2, 3, or 4 of these positions are further mutated, for example the amino acid at position 87 and/or 95 of SEQ ID NO: 5 can be changed to a V, A, S or T. In some examples, the amino acid at position 15 and/or 95 of SEQ ID NO: 5 can be changed to a V, A, or F. In some examples, combinations of these changes are made.

In some examples, the mutant FGF1 protein having a mutation at S116 (such as S116R) further includes one or more (such as 2, 3, 4, 5 or 6) of K12V, R35E/V, L46V, E87V/H, Y94V/F/A, N95V/T, C117V/A, K118N, K118E/V, and P134V (wherein the numbering refers to SEQ ID NO: 5). In some examples, the point mutation includes replacing amino acid sequence ILFLPLPV (amino acids 145-152 of SEQ ID NOS: 2 and 4) to AAALPLPV, ILALPLPV, ILFAPLPV, or ILFLPAPA. In some examples, such an FGF1 protein having a mutation at S116 (such as S116R) with one or more additional point mutations has reduced mitogenic activity as compared to wild-type mature FGF1 protein. In some examples, the mutant FGF1 protein includes R35E or R35V, (wherein the numbering refers to SEQ ID NO: 5). Examples of FGF1 mutant proteins containing point mutations include, but are not limited to, the protein sequence shown in SEQ ID NOS: 11, 12, 13, 14, 15, 16, 17, 18, 19, 20, 21, 22, 23 and 24.

In some examples, the mutant FGF1 protein containing an S116 mutation (such as S116R) further includes mutations at residues that interact with the FGF1 receptor, such as Y15, E87, Y94, and N95. Thus, in some examples, 1, 2, 3, or 4 of these positions are further mutated, for example the amino acid at position 87 and/or 95 of SEQ ID NO: 5 can be changed to a V, A, S or T. In some examples, the amino acid at position 15 and/or 95 of SEQ ID NO: 5 can be changed to a V, A, or F. In some examples, combinations of these changes are made.

In some examples, FGF1 is further mutated to increase the thermostability of mature or truncated native FGF1, such as an increase of at least 10%, at least 20%, at least 30%, at least 40%, or at least 50%. Exemplary mutations that can be used to increase the thermostability of mutated FGF1 include, but are not limited to, one or more of: K12, C117, P134, L44, C83, F132, M67, L73, V109, L111, A103, R119, Δ104-106, Δ120-122, Q40, H93, and S47, wherein the numbering refers to SEQ ID NO: 5 (e.g., see Xia et al., PLoS One. 7:e48210, 2012). In some examples, thermostability of FGF1 is increased by using one or more of the following mutations: Q40P and S47I or Q40P, S47I, and H93G (or any other combination of these mutations).

In some examples, the mutant FGF1 protein containing an S116 mutation (such as S116R) further includes both an N-terminal truncation and one or more additional point mutations. Specific exemplary FGF1 mutant proteins containing an S116 mutation (such as S116R) are shown in SEQ ID NOS: 11, 13, 14, 15, 16, 17, 18, 19, 20, 21, 22, 23 or 24. In some examples, the FGF1 mutant protein includes at least 80% sequence identity to SEQ ID NOS: 11, 12, 13, 14, 15, 16, 17, 18, 19, 20, 21, 22, 23, or 24. Thus, the FGF1 mutant protein containing an S116 mutation (such as S116R) can have at least 90%, at least 95%, at least 96%, at least 97%, at least 98% or at least 99% sequence identity to SEQ ID NOS: 11, 13, 14, 15, 16, 17, 18, 19, 20, 21, 22, 23 or 24. In some examples, the FGF1 mutant protein containing an S116 mutation (such as S116R) includes or consists of SEQ ID NOS: 11, 13, 14, 15, 16, 17, 18, 19, 20, 21, 22, 23, or 24. The disclosure encompasses variants of the disclosed FGF1 mutant proteins containing an S116 mutation (such as S116R), such as variants of SEQ ID NOS: 11, 13, 14, 15, 16, 17, 18, 19, 20, 21, 22, 23, or 24 having 1 to 20, 1 to 15, 1 to 10, 1 to 8, 2 to 10, 1 to 5, 1 to 6, 2 to 12, 3 to 12, 5 to 12, or 5 to 10 additional mutations, such as conservative amino acid substitutions.

In some examples, the mutant FGF1 protein containing an S116 mutation (such as S116R) has at its N-terminus a methionine. In some examples, the mutant FGF1 protein containing an S116 mutation (such as S116R) is at least 120 amino acids in length, such as at least 125, at least 130, at least 135, at least 140, at least 145, at least 150, at least 155, at least 160, or at least 175 amino acids in length, such as 120-160, 125-160, 130-160, 150-160, 130-200, 130-180, 130-170, or 120-160 amino acids in length.

Exemplary N-terminally truncated FGF1 sequences and FGF1 point mutations that can be used to further modify an FGF1 mutant protein containing an S116 mutation (such as S116R) are shown in Tables 1 and 2 (as well as those specifically provided in any of SEQ ID NOS: 11, 13, 14, 15, 16, 17, 18, 19, 20, 21, 22, 23, or 24). One skilled in the art will appreciate that an FGF1 mutant protein containing an S116 mutation (such as S116R) can be further modified to include any N-terminal truncation in Table 2 (as well as those provided in any of SEQ ID NOS: 6, 7, 8, 9, 14, 19, 21, 22, 23, and 24) and/or any FGF1 point mutation in Table 1 or Table 2, and that such an FGF1 mutant protein can be used directly. In addition, mutations can be made to the sequences shown in Table 2, such as one or more of the mutations discussed herein (such as 1, 2, 3, 4, 5, 6, 7, 8, 9, 10, 11, 12, 13, 14, 15, 16, 17, 18, 19, or 20 amino acid substitutions, such as conservative amino acid substitutions, deletions, and/or additions).

TABLE 2
Exemplary mutations that can be used to generate an FGF1 mutant protein

| FGF1 Point Mutations | FGF1 Fragments |
|---|---|
| SEQ ID NO: 13 | SEQ ID NO: 6 |
| SEQ ID NO: 14 | SEQ ID NO: 7 |
| SEQ ID NO: 15 | SEQ ID NO: 8 |
| SEQ ID NO: 16 | SEQ ID NO: 9 |
| SEQ ID NO: 17 | |
| SEQ ID NO: 18 | |
| SEQ ID NO: 19 | |
| SEQ ID NO: 20 | |
| SEQ ID NO: 21 | |
| SEQ ID NO: 22 | |

Exemplary mutant FGF1 proteins containing an S116 mutation (such as S116R) are provided in SEQ ID NOS: 11, 13, 14, 15, 16, 17, 18, 19, 20, 21, 22, 23, and 24. One skilled in the art will recognize that minor variations can be made to these sequences, without adversely affecting the function of the protein (such as its ability to reduce blood glucose). For example, variants of the mutant FGF1 proteins containing an S116 mutation (such as S116R) include those having at least 90%, at least 95%, at least 96%, at least 97%, at least 98%, or at least 99% sequence identity to SEQ ID NO: 11, 13, 14, 15, 16, 17, 18, 19, 20, 21, 22, 23, or 24 (but are not a native FGF1 sequence, e.g., SEQ ID NO: 5), but retain the ability to treat a metabolic disease, or decrease blood glucose in a mammal (such as a mammal with type II diabetes). Thus, variants of SEQ ID NO: 11, 13, 14, 15, 16, 17, 18, 19, 20, 21, 22, 23, or 24 retaining at least 90%, at least 95%, at least 96%, at least 97%, at least 98%, or at least 99% sequence identity are of use in the disclosed methods.

The exemplary FGF1 mutant proteins containing an S116 mutation (such as S116R) shown in SEQ ID NOS: 11, 13, 14, 15, 16, 17, 18, 19, 20, 21, 22, 23, and 24 can be further modified to be a chimeric protein. For example, any of SEQ ID NOS: 11, 13, 14, 15, 16, 17, 18, 19, 20, 21, 22, 23, and 24 can be further mutated to include at the N-terminus, C-terminus, or both, a portion of an FGF21 sequence, a portion of an FGF19 sequence, a β-Klotho-binding sequence, and/or an FGFR1c-binding sequence. Specific exemplary β-Klotho-binding/FGFR1c-binding chimeras that can be linked directly or indirectly to an N- or C-terminal end of a FGF1 mutant protein are shown in SEQ ID NOS: 74, 75, 76, and 77. For example, the C-terminal end or the N-terminal end of the disclosed FGF1 mutants can be joined directly or indirectly to the N-terminal end of a C-terminal fragment of FGF21 or FGF19, such as SEQ ID NO: 25 or 26, respectively. Similarly, the C-terminal end of the disclosed FGF1 mutants can be joined directly or indirectly to the N-terminal end of a β-Klotho binding domain (such as SEQ ID NOS: 27, 28, 29, 30, 31, 32, 33, 34, 35, 36, 37, 38, 39, 40, 41, 42, 43, 44, 45, 46, 47, 48, 49, 50, 51, 52 or β-Klotho binding portion of SEQ ID NO: 74, 75, 76, and 77), or the N-terminal end of the disclosed FGF1 mutants can be joined directly or indirectly to the C-terminal end of a β-Klotho-binding domain. In addition, the C-terminal end of the disclosed FGF1 mutants can be joined directly or indirectly to the N-terminal end of a FGFR1c-binding domain (such as SEQ ID NOS: 53, 54, 55, 56, 57, 58, 59, 60, 61, 62, 63, 64, 65, 66, 67, 68, 69, 70, 71, 72, 73 and 78), or the N-terminal end of the disclosed FGF1 mutants can be joined directly or indirectly to the C-terminal end of a FGFR1c-binding domain. In some examples, the C-terminal end of the disclosed FGF1 mutants can be joined directly or indirectly to an FGFR1c-binding domain (such as any of SEQ ID NOS: 53, 54, 55, 56, 57, 58, 59, 60, 61, 62, 63, 64, 65, 66, 67, 68, 69, 70, 71, 72, 73 or FGFR1c-binding portion of SEQ ID NOS: 74, 75, 76, and 77) and a β-Klotho-binding domain, the N-terminal end of the disclosed FGF1 mutants can be joined directly or indirectly to the C-terminal end of a FGFR1c-binding domain and a β-Klotho-binding domain, or both (such as SEQ ID NOS: 74, 75, 76, and 77). Such chimeric proteins can be used in the methods provided herein, for example to reduce blood glucose in a mammal, for example to treat a metabolic disease.

### FGF1

FGF1 (such as SEQ ID NOS: 2, 4 or 5) containing an S116 mutation (such as S116R) may further include mutations to control (e.g., reduce) the mitogenicity of the protein (for example by mutating the nuclear localization sequence (NLS) or the heparan sulfate binding region or both) and to provide glucose-lowering ability to the protein. Mutations can also be introduced to affect the stability and receptor binding selectivity of the protein.

Exemplary full-length FGF1 proteins are shown in SEQ ID NOS: 2 (human) and 4 (mouse). In some examples, FGF1 includes SEQ ID NOS: 2 or 4, but without the N-terminal methionine (resulting in a 154 aa FGF1 protein). In addition, the mature/active form of FGF1 is one where a portion of the N-terminus is removed, such as the N-terminal 15, 16, 20, or 21 amino acids from SEQ ID NOS: 2 or 4. Thus, in some examples the active form of FGF1 comprises or consists of amino acids 16-155 or 22-155 of SEQ ID NOS: 2 or 4 (e.g., see SEQ ID NO: 5). In some examples, the mature form of FGF1 that can be mutated includes SEQ ID NO: 5 with a methionine added to the N-terminus (wherein such a sequence can be mutated as discussed herein). Thus, a mutated mature FGF1 protein containing an S116 mutation (such as S116R) can include an N-terminal truncation.

In some examples, multiple types of mutations disclosed herein are made to an FGF1 protein. Although mutations below are noted by a particular amino acid for example in SEQ ID NOS: 2, 4, or 5, one skilled in the art will appreciate that the corresponding amino acid can be mutated in any FGF1 sequence. For example, Q40 of SEQ ID NO: 5 corresponds to Q55 of SEQ ID NO: 2 and 4.

In one example, mutations are made to the N-terminal region of FGF1 (such as SEQ ID NOS: 2, 4, or 5) containing an S116 mutation (such as S116R), such as deletion of the first 14, 15, 16, 17, 18, 19, 20, 21, 22, 23, 24, 25, 26, 27, 28, 29, or 30 amino acids of SEQ ID NOS: 2 or 4 (such as deletion of at least the first 14 amino acids of SEQ ID NOS: 2 or 4, such as deletion of at least the first 15, at least 16, at least 20, at least 25, or at least 29 amino acids of SEQ ID NOS: 2 or 4), deletion of the first 8, 9, 10, 11, 12, 13, 14, 15, 16, 17, 18, 19, or 20 amino acids of SEQ ID NO: 5 (e.g., see SEQ ID NOS: 7, 8 and 9).

Mutations can be made to a mutant FGF1 containing an S116 mutation (such as S116R) (such as to SEQ ID NOS: 11, 13, 14, 15, 16, 17, 18, 19, 20, 21, 22, 23, or 24) to reduce its mitogenic activity. In some examples, such mutations reduce mitogenic activity by at least 50%, at least 60%, at least 70%, at least 75%, at least 80%, at least 90%, at least 92%, at least 95%, at least 98%, at least 99%, or even complete elimination of detectable mitogenic activity, as compared to a native FGF1 protein without the mutation. Methods of measuring mitogenic activity are known in the art, such as thymidine incorporation into DNA in serum-starved cells (e.g., NIH 3T3 cells) stimulated with the mutated FGF1, methylthiazoletetrazolium (MTT) assay (for example by stimulating serum-starved cells with mutated FGF1 for 24 hr then measuring viable cells), cell number quantification or BrdU incorporation. In some examples, the assay provided by Fu et al., World J. Gastroenterol. 10:3590-6, 2004; Klingenberg et al., J. Biol. Chem. 274:18081-6, 1999; Shen et al., Protein Expr Purif 81:119-25, 2011, or Zou et al., Chin. Med. J. 121:424-429, 2008 is used to measure mitogenic activity. Examples of such mutations include, but are not limited to K12V, R35E, L46V, E87V, N95V, K12V/N95V, and Lys12Val/Pro134Val, Lys12Val/Leu46Val/Glu87Val/Asn95Val/Pro134Val (wherein the numbering refers to the sequence shown SEQ ID NO: 5). In some examples, a portion of contiguous N-terminal residues are removed, such as amino acids 1-9 of SEQ ID NO: 5, to produce a non-mitogenic form of FGF1. An example is shown in SEQ ID NO: 9.

Mutations that reduce the heparan binding affinity (such as a reduction of at least 10%, at least 20%, at least 50%, or at least 75%, e.g., as compared to a native FGF1 protein without the mutation), can also be used to reduce mitogenic activity, for example by substituting heparan binding residues from a paracrine FGFs into mutant FGF1 containing an S116 mutation (such as S116R).

Additional mutations can also be introduced into one or both nuclear localization sites (NLS1, amino acids 24-27 of SEQ ID NO: 2 and NLS2, amino acids 115-128 of SEQ ID NO: 4) of FGF1, for example to reduce mitogenicity, as compared to a native FGF1 protein without the mutation. Examples of NLS mutations that can be made to a mutant FGF1 containing an S116 mutation (such as S116R) include, but are not limited to: deleting or mutating all or a part of NLS1 (such as deleting or mutating the lysines), deleting or mutating the lysines in NLS2 such as ¹¹⁵KK . . . ¹²⁷KK . . . , or combinations thereof (wherein the numbering refers to the sequence shown SEQ ID NO: 2). For example, one or more of 24K, 25K, 27K, 115K, 127K or 128K (wherein the numbering refers to the sequence shown SEQ ID NO: 2) or can be mutated (for example changed to an alanine or deleted). Particular examples of such mutations that can be made to the heparan binding site in the NLS2 (KKN . . . KR) include K118N or K118E).

Mutations can be introduced into the phosphorylation site of an FGF1 mutant containing an S116 mutation (such as S116R), for example to create a constitutively active or inactive mutant to affect nuclear signaling.

In some examples, an FGF1 mutant containing an S116 mutation (such as S116R) includes additional mutations to the FGF1 nuclear export sequence, for example to decrease the amount of FGF1 in the nucleus and reduce its mitogenicity as measured by thymidine incorporation assays in cultured cells (e.g., see Nilsen et al., J. Biol. Chem. 282(36):26245-56, 2007). Mutations to the nuclear export sequence decrease FGF1-induced proliferation (e.g., see Nilsen et al., J. Biol. Chem. 282(36):26245-56, 2007). Methods of measuring FGF1 degradation are known in the art, such as measuring [³⁵S]methionine-labeled FGF1 or immunoblotting for steady-state levels of FGF1 in the presence or absence of proteasome inhibitors. In one example, the assay provided by Nilsen et al., J. Biol. Chem. 282(36):26245-56, 2007 or Zakrzewska et al., J. Biol. Chem. 284:25388-403, 2009 is used to measure FGF1 degradation.

The FGF1 nuclear export sequence includes amino acids 145-152 of SEQ ID NOS: 2 and 4 or amino acids 130-137 of SEQ ID NO: 5. Examples of FGF1 nuclear export sequence mutations that can be made to a mutant FGF1 containing an S116 mutation (such as S116R) include, but are not limited to, changing the sequence ILFLPLPV (amino acids 145-152 of SEQ ID NOS: 2 and 4) to AAALPLPV, ILALPLPV, ILFAPLPV, or ILFLPAPA.

In one example, mutations are introduced to improve stability of a mutant FGF1 containing an S116 mutation (such as S116R). In some examples, the sequence NYKKPKL (amino acids 22-28 of SEQ ID NO: 2) is not altered, and in some examples ensures for structural integrity of FGF1 and increases interaction with the FGF1 receptor. Methods of measuring FGF1 stability are known in the art, such as measuring denaturation of FGF1 or mutants by fluorescence and circular dichroism in the absence and presence of a 5-fold molar excess of heparin in the presence of 1.5 M urea or isothermal equilibrium denaturation by guanidine hydrochloride. In one example, the assay provided by Dubey et al., J. Mol. Biol. 371:256-268, 2007 is used to measure FGF1 stability. Examples of mutations that can be used to increase stability of the protein include, but are not limited to, one or more of Q40P, S47I and H93G (wherein the numbering refers to the sequence shown SEQ ID NO: 5).

In one example, mutations are introduced to improve the thermostability of FGF1 containing an S116 mutation (such as S116R), such as an increase of at least 10%, at least 20%, at least 50%, or at least 75%, as compared to the FGF1 protein without the additional mutation (e.g., see Xia et al., PLoS One. 2012; 7(11):e48210 and Zakrzewska, J Biol Chem. 284:25388-25403, 2009). In one example, mutations are introduced to increase protease resistance of FGF1 containing an S116 mutation (such as S116R) (e.g., see Kobielak et al., Protein Pept Lett. 21(5):434-43, 2014). Other mutations that can be made to FGF1 containing an S116 mutation (such as S116R) include those mutations provided in Lin et al., J Biol Chem. 271(10):5305-8, 1996).

In some examples, the mutant FGF1 protein containing an S116 mutation (such as S116R) is PEGylated at one or more positions, such as at N95 (for example see methods of Niu et al., J. Chromatog. 1327:66-72, 2014, herein incorporated by reference). Pegylation consists of covalently linking a polyethylene glycol group to surface residues and/or the N-terminal amino group. N95 is known to be involved in receptor binding, and thus, is on the surface of the folded protein. As mutations to surface exposed residues could potentially generate immunogenic sequences, pegylation is an alternative method to abrogate a specific interaction. Pegylation is an option for any surface exposed site implicated in the receptor binding and/or proteolytic degradation. Pegylation can “cover” functional amino acids, e.g. N95, as well as increase serum stability.

In some examples, the mutant FGF1 protein includes an immunoglobin FC domain (for example see Czajkowsky et al., EMBO Mol. Med. 4:1015-28, 2012, herein incorporated by reference). The conserved FC fragment of an antibody can be incorporated either N-terminal or C-terminal of the mutant FGF1 protein, and can enhance stability of the protein and therefore serum half-life. The FC domain can also be used as a means to purify the proteins on Protein A or Protein G sepharose beads. This makes the FGF1 mutants having heparin binding mutations easier to purify.

### Variant Sequences

Variant FGF1 proteins containing an S116 mutation (such as S116R), including variants of the sequences shown in Tables 1 and 2, and variants of SEQ ID NOS: 11, 13, 14, 15, 16, 17, 18, 19, 20, 21, 22, 23, or 24, can contain one or more mutations, such as a single insertion, a single deletion, a single substitution. In some examples, the mutant FGF1 protein containing an S116 mutation (such as S116R) includes 1-20 insertions, 1-20 deletions, 1-20 substitutions, and/or any combination thereof (e.g., single insertion together with 1-19 substitutions). In some examples, the disclosure provides a variant of any disclosed mutant FGF1 protein containing an S116 mutation (such as S116R) having 1, 2, 3, 4, 5, 6, 7, 8, 9, 10, 11, 12, 13, 14, 15, 16, 17, 18, 19 or 20 additional amino acid changes. In some examples, SEQ ID NOS: 11, 13, 14, 15, 16, 17, 18, 19, 20, 21, 22, 23 or 24, includes 1-8 insertions, 1-15 deletions, 1-10 substitutions, or any combination thereof (e.g., 1-15, 1-4, or 1-5 amino acid deletions together with 1-10, 1-5 or 1-7 amino acid substitutions). In some examples, the disclosure provides a variant of SEQ ID NOS: 11, 13, 14, 15, 16, 17, 18, 19, 20, 21, 22, 23, or 24, having 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid changes. In one example, such variant peptides are produced by manipulating the nucleotide sequence encoding a peptide using standard procedures such as site-directed mutagenesis or PCR. Such variants can also be chemically synthesized.

One type of modification or mutation includes the substitution of amino acids for amino acid residues having a similar biochemical property, that is, a conservative substitution (such as 1-4, 1-8, 1-10, or 1-20 conservative substitutions). Typically, conservative substitutions have little to no impact on the activity of a resulting peptide. For example, a conservative substitution is an amino acid substitution in SEQ ID NOS: 11, 12, 13, 14, 15, 16, 17, 18, 19, 20, 21, 22, 23, or 24 that does not substantially affect the ability of the peptide to decrease blood glucose in a mammal. An alanine scan can be used to identify which amino acid residues in a mutant FGF1 protein containing an S116 mutation (such as S116R), such as SEQ ID NO: 11, 13, 14, 15, 16, 17, 18, 19, 20, 21, 22, 23, or 24, can tolerate an amino acid substitution. In one example, the blood glucose lowering activity of FGF1, or any of SEQ ID NOS: 11, 12, 13, 14, 15, 16, 17, 18, 19, 20, 21, 22, 23, and 24 is not altered by more than 25%, for example not more than 20%, for example not more than 10%, when an alanine, or other conservative amino acid, is substituted for 1-4, 1-8, 1-10, or 1-20 native amino acids. Examples of amino acids which may be substituted for an original amino acid in a protein and which are regarded as conservative substitutions include: Ser for Ala; Lys, Gln, or Asn for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

More substantial changes can be made by using substitutions that are less conservative, e.g., selecting residues that differ more significantly in their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in polypeptide function are those in which: (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine. The effects of these amino acid substitutions (or other deletions and/or additions) can be assessed by analyzing the function of the mutant FGF1 protein containing an S116 mutation (such as S116R), such as any of SEQ ID NOS: 11, 13, 14, 15, 16, 17, 18, 19, 20, 21, 22, 23, and 24 by analyzing the ability of the variant protein to decrease blood glucose in a mammal.

### Generation of Proteins

Isolation and purification of recombinantly expressed mutated FGF1 proteins can be carried out by conventional means, such as preparative chromatography and immunological separations. Once expressed, mutated FGF1 proteins can be purified according to standard procedures of the art, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes.

In addition to recombinant methods, mutated FGF1 proteins disclosed herein can also be constructed in whole or in part using standard peptide synthesis. In one example, mutated FGF1 proteins are synthesized by condensation of the amino and carboxyl termini of shorter fragments. Methods of forming peptide bonds by activation of a carboxyl terminal end (such as by the use of the coupling reagent N, N′-dicylohexylcarbodimide) are well known in the art.

### Mutated FGF1 Nucleic Acid Molecules and Vectors

Nucleic acid molecules encoding a mutated FGF1 protein containing an S116 mutation (such as S116R) are encompassed by this disclosure. Based on the genetic code, nucleic acid sequences coding for any mutated FGF1 sequence, such as those generated using the sequences shown in Tables 1 and 2, can be routinely generated. In some examples, such a sequence is optimized for expression in a host cell, such as a host cell used to express the mutant FGF1 protein.

In one example, a nucleic acid sequence codes for a mutant FGF1 protein containing an S116 mutation (such as S116R) having at least 60%, at least 70%, at least 75%, at least 80%, at least 90%, at least 92%, at least 95%, at least 96%, at least 97%, at least 99% or at least 99% sequence identity to SEQ ID NO: 11, 13, 14, 15, 16, 17, 18, 19, 20, 21, 22, 23, or 24 can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same mutant FGF1 protein sequence. In one example, a mutant FGF1 containing an S116 mutation (such as S116R) nucleic acid sequence has at least 70%, at least 80%, at least 85%, at least 90%, at least 92%, at least 95%, at least 98%, or at least 99% sequence identity to SEQ ID NO: 10.

Nucleic acid molecules include DNA, cDNA, and RNA sequences which encode a mutated FGF1 peptide. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3^{rd} Edition, W.H. 5 Freeman and Co., NY).

Codon preferences and codon usage tables for a particular species can be used to engineer isolated nucleic acid molecules encoding a mutated FGF1 protein containing an S116 mutation (such as S116R) (such as one encoding a protein generated using the sequences shown in Tables 1 and 2, the sequences in any of SEQ ID NOS: 11, 13, 14, 15, 16, 17, 18, 19, 20, 21, 22, 23 or 24, or those encoding a protein having at least 90%, at least 92%, at least 95%, at least 96%, at least 97%, at least 98%, at least 99%, or 100% sequence identity to SEQ ID NOS: 11, 13, 14, 15, 16, 17, 18, 19, 20, 21, 22, 23, or 24) that take advantage of the codon usage preferences of that particular species. For example, the mutated FGF1 proteins disclosed herein can be designed to have codons that are preferentially used by a particular organism of interest.

A nucleic acid encoding a mutant FGF1 protein containing an S116 mutation (such as S116R) (such as one encoding a protein generated using the sequences shown in Tables 1 and 2, the sequences in any of SEQ ID NOS: 11, 13, 14, 15, 16, 17, 18, 19, 20, 21, 22, 23, or 24 or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). A wide variety of cloning and in vitro amplification methodologies are well known to persons skilled in the art. In addition, nucleic acids encoding sequences encoding a mutant FGF1 protein containing an S116 mutation (such as S116R) (such as one encoding a protein generated using the sequences shown in Tables 1 and 2, the sequences in any of SEQ ID NOS: 11, 13, 14, 15, 16, 17, 18, 19, 20, 21, 22, 23, or 24 or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be prepared by cloning techniques. Examples of appropriate cloning and sequencing techniques, and instructions sufficient to direct persons of skill through cloning are found in Sambrook et al. (ed.), Molecular Cloning: A Laboratory Manual 2nd ed., vol. 1-3, Cold Spring Harbor Laboratory Press, Cold Spring, Harbor, N.Y., 1989, and Ausubel et al., (1987) in “Current Protocols in Molecular Biology,” John Wiley and Sons, New York, N.Y.

Nucleic acid sequences encoding a mutated FGF1 protein containing an S116 mutation (such as S116R) (such as one encoding a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is generally limited to sequences of about 100 bases, longer sequences may be obtained by the ligation of shorter sequences.

In one example, a mutant FGF1 protein containing an S116 mutation (such as S116R) (such as a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NOS: 11, 13, 14, 15, 16, 17, 18, 19, 20, 21, 22, 23, or 24) is prepared by inserting the cDNA which encodes the mutant FGF1 protein containing an S116 mutation (such as S116R) into a vector. The insertion can be made so that the mutant FGF1 protein is read in frame so that the mutant FGF1 protein is produced.

The mutated FGF1 protein containing an S116 mutation (such as S116R) nucleic acid coding sequence (such as one encoding a protein generated using the sequences shown in Tables 1 and 2, the sequences in any of SEQ ID NOS: 11, 13, 14, 15, 16, 17, 18, 19, 20, 21, 22, 23, or 24 or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect, plant, and mammalian organisms. Methods of expressing DNA sequences having eukaryotic or viral sequences in prokaryotes are well known in the art. Biologically functional viral and plasmid DNA vectors capable of expression and replication in a host are known in the art. The vector can encode a selectable marker, such as a thymidine kinase gene.

Nucleic acid sequences encoding a mutated FGF1 protein containing an S116 mutation (such as S116R) (such as one encoding a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be operatively linked to expression control sequences. An expression control sequence operatively linked to a mutated FGF1 protein coding sequence is ligated such that expression of the mutant FGF1 protein coding sequence is achieved under conditions compatible with the expression control sequences. The expression control sequences include, but are not limited to appropriate promoters, enhancers, transcription terminators, a start codon (i.e., ATG) in front of a mutated FGF1 protein-encoding gene, splicing signal for introns, maintenance of the correct reading frame of that gene to permit proper translation of mRNA, and stop codons.

In one embodiment, vectors are used for expression in yeast such as S. cerevisiae, P. pastoris, or Kluyveromyces lactis. Several promoters are known to be of use in yeast expression systems such as the constitutive promoters plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation. The nucleic acid molecules encoding a mutated FGF1 protein containing an S116 mutation (such as S116R) (such as one encoding a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can also be designed to express in insect cells.

A mutated FGF1 protein containing an S116 mutation (such as S116R) (such as a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast cell lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared that encode a mutated FGF1 protein containing an S116 mutation (such as S116R) (such as one encoding a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24). Exemplary viral vectors include polyoma, SV40, adenovirus, vaccinia virus, adeno-associated virus, herpes viruses including HSV and EBV, Sindbis viruses, alphaviruses and retroviruses of avian, murine, and human origin. Baculovirus (Autographa californica multinuclear polyhedrosis virus; AcMNPV) vectors are also known in the art, and may be obtained from commercial sources. Other suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus, and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox, and pigeon pox. Capripox include goatpox and sheeppox. In one example, the suipox is swinepox. Other viral vectors that can be used include other DNA viruses such as herpes virus and adenoviruses, and RNA viruses such as retroviruses and polio.

Viral vectors that encode a mutated FGF1 protein containing an S116 mutation (such as S116R) (such as one encoding a protein generated using the sequences shown in Tables 1 and 2, the sequences in SEQ ID NOS: 11, 13, 14, 15, 16, 17, 18, 19, 20, 21, 22, 23 or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can include at least one expression control element operationally linked to the nucleic acid sequence encoding the mutated FGF1 protein. The expression control elements are inserted in the vector to control and regulate the expression of the nucleic acid sequence. Examples of expression control elements of use in these vectors includes, but is not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the mutated FGF1 protein in the host system. The expression vector can contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

Basic techniques for preparing recombinant DNA viruses containing a heterologous DNA sequence encoding the mutated FGF1 protein containing an S116 mutation (such as S116R) (such as one encoding a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) are known. Such techniques involve, for example, homologous recombination between the viral DNA sequences flanking the DNA sequence in a donor plasmid and homologous sequences present in the parental virus. The vector can be constructed for example by steps known in the art, such as by using a unique restriction endonuclease site that is naturally present or artificially inserted in the parental viral vector to insert the heterologous DNA.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a mutated FGF1 protein containing an S116 mutation (such as S116R) (such as one encoding a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24), and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression systems such as plasmids and vectors of use in producing mutated FGF1 proteins in cells including higher eukaryotic cells such as the COS, CHO, HeLa and myeloma cell lines.

### Cells Expressing Mutated FGF1 Proteins

A nucleic acid molecule encoding a mutated FGF1 protein containing an S116 mutation (such as S116R) disclosed herein can be used to transform cells and make transformed cells. Thus, cells expressing a mutated FGF1 protein containing an S116 mutation (such as S116R) (such as a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24), are disclosed. Cells expressing a mutated FGF1 protein containing an S116 mutation (such as S116R) disclosed herein can be eukaryotic or prokaryotic. Examples of such cells include, but are not limited to bacteria, archea, plant, fungal, yeast, insect, and mammalian cells, such as Lactobacillus, Lactococcus, Bacillus (such as B. subtilis), Escherichia (such as E. coli), Clostridium, Saccharomyces or Pichia (such as S. cerevisiae or P. pastoris), Kluyveromyces lactis, Salmonella typhimurium, SF9 cells, C129 cells, 293 cells, Neurospora, and immortalized mammalian myeloid and lymphoid cell lines.

Cells expressing a mutated FGF1 protein containing an S116 mutation (such as S116R) are transformed or recombinant cells. Such cells can include at least one exogenous nucleic acid molecule that encodes a mutated FGF1 protein, for example one encoding a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24. It is understood that all progeny may not be identical to the parental cell since there may be mutations that occur during replication. Methods of stable transfer, meaning that the foreign DNA is continuously maintained in the host cell, are known in the art.

Transformation of a host cell with recombinant DNA may be carried out by conventional techniques. Where the host is prokaryotic, such as E. coli, competent cells which are capable of DNA uptake can be prepared from cells harvested after exponential growth phase and subsequently treated with CaCl₂. Alternatively, MgCl₂ or RbCl can be used. Transformation can also be performed after forming a protoplast of the host cell, or by electroporation. Techniques for the propagation of mammalian cells in culture are known (see, Jakoby and Pastan (eds.), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although other cell lines may be used, such as cells designed to provide higher expression desirable glycosylation patterns, or other features. Techniques for the transformation of yeast cells, such as polyethylene glycol transformation, protoplast transformation, and gene guns are also known in the art.

### Pharmaceutical Compositions that Include Mutated FGF1 Molecules

Pharmaceutical compositions that include a mutated FGF1 protein containing an S116 mutation (such as S116R) (such as a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NOS: 11, 13, 14, 15, 16, 17, 18, 19, 20, 21, 22, 23, or 24) or a nucleic acid encoding these proteins, can be formulated with an appropriate pharmaceutically acceptable carrier, depending upon the particular mode of administration chosen.

In some embodiments, the pharmaceutical composition consists essentially of a mutated FGF1 protein containing an S116 mutation (such as S116R) (such as a protein generated using the sequences shown in Table 1 or 2, the sequences in any of SEQ ID NOS: 11, 13, 14, 15, 16, 17, 18, 19, 20, 21, 22, 23, or 24, or a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) (or a nucleic acid encoding such a protein) and a pharmaceutically acceptable carrier. In these embodiments, additional therapeutically effective agents are not included in the compositions.

In other embodiments, the pharmaceutical composition includes a mutated FGF1 protein containing an S116 mutation (such as S116R) (such as a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) (or a nucleic acid encoding such a protein) and a pharmaceutically acceptable carrier. Additional therapeutic agents, such as agents for the treatment of diabetes, can be included. Thus, the pharmaceutical compositions can include a therapeutically effective amount of another agent. Examples of such agents include, without limitation, anti-apoptotic substances such as the Nemo-Binding Domain and compounds that induce proliferation such as cyclin dependent kinase (CDK)-6, CDK-4 and cyclin D1. Other active agents can be utilized, such as antidiabetic agents for example, insulin, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-gamma-agonists (such as C1262570, aleglitazar, farglitazar, muraglitazar, tesaglitazar, and TZD) and PPAR-γ antagonists, PPAR-gamma/alpha modulators (such as KRP 297), alpha-glucosidase inhibitors (e.g., acarbose, voglibose), dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g. exendin-4) or amylin. Additional examples include immunomodulatory factors such as anti-CD3 mAb, growth factors such as HGF, VEGF, PDGF, lactogens, and PTHrP. In some examples, the pharmaceutical compositions containing a mutated FGF1 protein can further include a therapeutically effective amount of other FGFs, such as FGF21, FGF19, or both, heparin, or combinations thereof.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21′ Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some embodiments, a mutated FGF1 protein containing an S116 mutation (such as S116R) (such as a protein generated using the sequences shown in Tables 1 and 2, the sequences in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) is included in a controlled release formulation, for example, a microencapsulated formulation. Various types of biodegradable and biocompatible polymers, methods can be used, and methods of encapsulating a variety of synthetic compounds, proteins and nucleic acids, have been well described in the art (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2^{nd} ed., CRC Press, 2006).

In other embodiments, a mutated FGF1 protein containing an S116 mutation (such as S116R) (such as a protein generated using the sequences shown in Tables 1 and 2, the sequences in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) is included in a nanodispersion system. Nanodispersion systems and methods for producing such nanodispersions are well known to one of skill in the art. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006.

With regard to the administration of nucleic acids, one approach to administration of nucleic acids is direct treatment with plasmid DNA, such as with a mammalian expression plasmid. As described above, the nucleotide sequence encoding a mutated FGF1 protein containing an S116 mutation (such as S116R) (such as a protein generated using the sequences shown in Tables 1 and 2, the sequences in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be placed under the control of a promoter to increase expression of the protein.

Many types of release delivery systems are available and known. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which a mutated FGF1 protein containing an S116 mutation (such as S116R) (such as a protein generated using the sequences shown in Tables 1 and 2, the sequences in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24), or polynucleotide encoding this protein, is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Use of a long-term sustained release implant may be particularly suitable for treatment of chronic conditions, such as diabetes. Long-term release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. These systems have been described for use with nucleic acids (see U.S. Pat. No. 6,218,371). For use in vivo, nucleic acids and peptides are preferably relatively resistant to degradation (such as via endo- and exo-nucleases). Thus, modifications of the disclosed mutated FGF1 proteins, such as the inclusion of a C-terminal amide, can be used.

The dosage form of the pharmaceutical composition can be determined by the mode of administration chosen. For instance, in addition to injectable fluids, topical, inhalation, oral, and suppository formulations can be employed. Topical preparations can include eye drops, ointments, sprays, patches, and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The pharmaceutical compositions that include a mutated FGF1 protein containing an S116 mutation (such as S116R) (such as a protein generated using the sequences shown in Tables 1 and 2, the sequence in SEQ ID NO: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be formulated in unit dosage form, suitable for individual administration of precise dosages. In one non-limiting example, a unit dosage contains from about 1 mg to about 1 g of a mutated FGF1 protein containing an S116 mutation (such as S116R) (such as a protein generated using the sequences shown in Tables 1 and 2, the sequences in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24), such as about 10 mg to about 100 mg, about 50 mg to about 500 mg, about 100 mg to about 900 mg, about 250 mg to about 750 mg, or about 400 mg to about 600 mg. In other examples, a therapeutically effective amount of a mutated FGF1 protein (such as a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) is about 0.01 mg/kg to about 50 mg/kg, for example, about 0.1 mg/kg to about 25 mg/kg, about 0.1 mg/kg to about 0.5 mg/kg, about 0.05 mg/kg to about 0.1 mg/kg, about 0.01 mg/kg to about 0.0 mg/kg, or about 1 mg/kg to about 10 mg/kg. In other examples, a therapeutically effective amount of a mutated FGF1 protein containing an S116 mutation (such as S116R) (such as a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) is about 1 mg/kg to about 5 mg/kg, for example about 2 mg/kg. In a particular example, a therapeutically effective amount of a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) includes about 1 mg/kg to about 10 mg/kg, such as about 2 mg/kg. In a particular example, a therapeutically effective amount of a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) includes about 0.01 mg/kg to about 0.5 mg/kg, such as about 0.1 mg/kg.

### Treatment Using a Mutated FGF1 Protein

The disclosed mutated FGF1 proteins containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24), or nucleic acids encoding such proteins, can be administered to a subject, for example to treat a metabolic disease, for example by reducing fed and fasting blood glucose, improving insulin sensitivity and glucose tolerance, reducing systemic chronic inflammation, ameliorating hepatic steatosis in a mammal, reducing food intake, or combinations thereof.

The compositions of this disclosure that include a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) (or nucleic acids encoding these molecules) can be administered to humans or other animals by any means, including orally, intravenously, intramuscularly, intraperitoneally, intranasally, intradermally, intrathecally, subcutaneously, via inhalation or via suppository. In one non-limiting example, the composition is administered via injection. In some examples, site-specific administration of the composition can be used, for example by administering a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) (or a nucleic acid encoding these molecules) to pancreas tissue (for example by using a pump, or by implantation of a slow release form at the site of the pancreas). The particular mode of administration and the dosage regimen will be selected by the attending clinician, taking into account the particulars of the case (e.g. the subject, the disease, the disease state involved, the particular treatment, and whether the treatment is prophylactic). Treatment can involve daily or multi-daily or less than daily (such as weekly, every other week, monthly, every 7 days, every 10 days, every 14 days, every 30 days, etc.) doses over a period of a few days, few weeks, to months, or even years. For example, a therapeutically effective amount of a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be administered in a single dose, twice daily, weekly, every other week, or in several doses, for example daily, or during a course of treatment. In a particular non-limiting example, treatment involves once daily dose, twice daily dose, once weekly dose, every other week dose, or monthly dose.

The amount of a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NOS: 11, 13, 14, 15, 16, 17, 18, 19, 20, 21, 22, 23, or 24) administered can be dependent on the subject being treated, the severity of the affliction, and the manner of administration, and is best left to the judgment of the prescribing clinician. Determination of the appropriate amount to be administered is within the routine level of skill in the art. Within these bounds, the formulation to be administered will contain a quantity of the mutated FGF1 protein in amounts effective to achieve the desired effect in the subject being treated. A therapeutically effective amount of a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23 or 24. or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be the amount of the mutant FGF1 protein or a nucleic acid encoding these molecules that is necessary to treat diabetes or reduce blood glucose levels (for example a reduction of at least 5%, at least 10% or at least 20%, for example relative to no administration of the mutant FGF1).

When a viral vector is utilized for administration of an nucleic acid encoding a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24 or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24), the recipient can receive a dosage of each recombinant virus in the composition in the range of from about 10⁵ to about 10¹⁰ plaque forming units/mg mammal, although a lower or higher dose can be administered. Examples of methods for administering the composition into mammals include, but are not limited to, exposure of cells to the recombinant virus ex vivo, or injection of the composition into the affected tissue or intravenous, subcutaneous, intradermal or intramuscular administration of the virus. Alternatively the recombinant viral vector or combination of recombinant viral vectors may be administered locally by direct injection into the pancreas in a pharmaceutically acceptable carrier.

Generally, the quantity of recombinant viral vector, carrying the nucleic acid sequence of the mutated FGF1 protein containing an S116 mutation (such as S116R) to be administered (such as a protein generated using the sequences shown in Tables 1 and 2, the sequence in SEQ ID NOS: 11, 13, 14, 15, 16, 17, 18, 19, 20, 21, 22, 23, or 24 or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) is based on the titer of virus particles. An exemplary range to be administered is 10⁵ to 10¹⁰ virus particles per mammal, such as a human.

In some examples, a mutated FGF1 protein containing an S116 mutation (such as S116R) (such as a protein generated using the sequences shown in Tables 1 and 2, the sequences in any of SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24), or a nucleic acid encoding the mutated FGF1 protein, is administered in combination (such as sequentially or simultaneously or contemporaneously) with one or more other agents, such as those useful in the treatment of diabetes or insulin resistance.

Anti-diabetic agents are generally categorized into six classes: biguanides (e.g., metformin); thiazolidinediones (including rosiglitazone (Avandia®), pioglitazone (Actos®), rivoglitazone, and troglitazone); sulfonylureas; inhibitors of carbohydrate absorption; fatty acid oxidase inhibitors and anti-lipolytic drugs; and weight-loss agents. Any of these agents can also be used in the methods disclosed herein. The anti-diabetic agents include those agents disclosed in Diabetes Care, 22(4):623-634. One class of anti-diabetic agents of use is the sulfonylureas, which are believed to increase secretion of insulin, decrease hepatic glucogenesis, and increase insulin receptor sensitivity. Another class of anti-diabetic agents is the biguanide antihyperglycemics, which decrease hepatic glucose production and intestinal absorption, and increase peripheral glucose uptake and utilization, without inducing hyperinsulinemia.

In some examples, a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) can be administered in combination with effective doses of anti-diabetic agents (such as biguanides, thiazolidinediones, or incretins) and/or lipid lowering compounds (such as statins or fibrates). The terms “administration in combination,” “co-administration,” or the like, refer to both concurrent and sequential administration of the active agents. Administration of a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24 or those encoding a protein having at least 90%, at least 92%, at least 95%, at least 96%, at least 97%, at least 98%, at least 99%, or 100% sequence identity to SEQ ID NOS: 11, 13, 14, 15, 16, 17, 18, 19, 20, 21, 22, 23, or 24) or a nucleic acid encoding such a mutant FGF1 protein, may also be in combination with lifestyle modifications, such as increased physical activity, low fat diet, low sugar diet, and smoking cessation.

Additional agents that can be used in combination with the disclosed mutated FGF1 proteins include, without limitation, anti-apoptotic substances such as the Nemo-Binding Domain and compounds that induce proliferation such as cyclin dependent kinase (CDK)-6, CDK-4 and Cyclin D1. Other active agents can be utilized, such as antidiabetic agents for example, insulin, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-gamma-agonists (such as C1262570) and antagonists, PPAR-gamma/alpha modulators (such as KRP 297), alpha-glucosidase inhibitors (e.g., acarbose, voglibose), Dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g., exendin-4) or amylin. In some embodiments the agent is an immunomodulatory factor such as anti-CD3 mAb, growth factors such as HGF, vascular endothelial growth factor (VEGF), platelet derived growth factor (PDGF), lactogens, or parathyroid hormone related protein (PTHrP). In one example, the mutated FGF1 protein is administered in combination with a therapeutically effective amount of another FGF, such as FGF21, FGF19, or both, heparin, or combinations thereof.

In some embodiments, methods are provided for treating diabetes or pre-diabetes in a subject by administering a therapeutically effective amount of a composition including or a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24 or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24), or a nucleic acid encoding the mutated FGF1 protein, to the subject. The subject can have diabetes type I or diabetes type II. The subject can be any mammalian subject, including human subjects and veterinary subjects such as cats and dogs. The subject can be a child or an adult. The subject can also be administered insulin. The method can include measuring blood glucose levels.

In some examples, the method includes selecting a subject with diabetes, such as type I or type II diabetes, or a subject at risk for diabetes, such as a subject with pre-diabetes. These subjects can be selected for treatment with the disclosed mutated FGF1 proteins containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24) or nucleic acid molecules encoding such.

In some examples, a subject with diabetes may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. In other examples, a subject with pre-diabetes may be diagnosed by impaired glucose tolerance (IGT). An OGTT two-hour plasma glucose of greater than or equal to 140 mg/dL and less than 200 mg/dL (7.8-11.0 mM), or a fasting plasma glucose (FPG) concentration of greater than or equal to 100 mg/dL and less than 125 mg/dL (5.6-6.9 mmol/L), or HbA1c levels of greater than or equal to 5.7% and less than 6.4% (5.7-6.4%) is considered to be IGT, and indicates that a subject has pre-diabetes. Additional information can be found in Standards of Medical Care in Diabetes—2010 (American Diabetes Association, Diabetes Care 33:S11-61, 2010).

In some examples, the subject treated with the disclosed compositions and methods has HbA1C of greater than 6.5% or greater than 7%.

In some examples, treating diabetes includes one or more of increasing glucose tolerance (such as an increase of at least 5%, at least 10%, at least 20%, or at least 50%, for example relative to no administration of the mutant FGF1 containing an S116 mutation (such as S116R)), decreasing insulin resistance (for example, decreasing plasma glucose levels, decreasing plasma insulin levels, or a combination thereof, such as decreases of at least 5%, at least 10%, at least 20%, or at least 50%, for example relative to no administration of the mutant FGF1), decreasing serum triglycerides (such as a decrease of at least 10%, at least 20%, or at least 50%, for example relative to no administration of the mutant FGF1 containing an S116 mutation (such as S116R)), decreasing free fatty acid levels (such as a decrease of at least 5%, at least 10%, at least 20%, or at least 50%, for example relative to no administration of the mutant FGF1 containing an S116 mutation (such as S116R)), and decreasing HbA1c levels in the subject (such as a decrease of at least 0.5%, at least 1%, at least 1.5%, at least 2%, or at least 5% for example relative to no administration of the mutant FGF1 containing an S116 mutation (such as S116R)). In some embodiments, the disclosed methods include measuring glucose tolerance, insulin resistance, plasma glucose levels, plasma insulin levels, serum triglycerides, free fatty acids, and/or HbA1c levels in a subject.

In some examples, administration of a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NOS: 11, 13, 14, 15, 16, 17, 18, 19, 20, 21, 22, 23, or 24), or nucleic acid molecule encoding such, treats a metabolic disease, such as diabetes (such as type II diabetes) or pre-diabetes, by decreasing of HbA1C, such as a reduction of at least 0.5%, at least 1%, or at least 1.5%, such as a decrease of 0.5% to 0.8%, 0.5% to 1%, 1 to 1.5% or 0.5% to 2%. In some examples the target for HbA1C is less than about 6.5%, such as about 4-6%, 4-6.4%, or 4-6.2%. In some examples, such target levels are achieved within about 26 weeks, within about 40 weeks, or within about 52 weeks. Methods of measuring HbA1C are routine, and the disclosure is not limited to particular methods. Exemplary methods include HPLC, immunoassays, and boronate affinity chromatography.

In some examples, administration of a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NOS: 11, 13, 14, 15, 16, 17, 18, 19, 20, 21, 22, 23, or 24), or nucleic acid molecule encoding such, treats diabetes or pre-diabetes by increasing glucose tolerance, for example, by decreasing blood glucose levels (such as two-hour plasma glucose in an OGTT or FPG) in a subject. In some examples, the method includes decreasing blood glucose by at least 5% (such as at least 10%, at least 15%, at least 20%, at least 25%, at least 30%, at least 35%, or more) as compared with a control (such as no administration of any of insulin, a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NOS: 11, 13, 14, 15, 16, 17, 18, 19, 20, 21, 22, 23, or 24), or a nucleic acid molecule encoding such). In particular examples, a decrease in blood glucose level is determined relative to the starting blood glucose level of the subject (for example, prior to treatment with a mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24), or nucleic acid molecule encoding such). In other examples, decreasing blood glucose levels of a subject includes reduction of blood glucose from a starting point (for example greater than about 126 mg/dL FPG or greater than about 200 mg/dL OGTT two-hour plasma glucose) to a target level (for example, FPG of less than 126 mg/dL or OGTT two-hour plasma glucose of less than 200 mg/dL). In some examples, a target FPG may be less than 100 mg/dL. In other examples, a target OGTT two-hour plasma glucose may be less than 140 mg/dL. Methods to measure blood glucose levels in a subject (for example, in a blood sample from a subject) are routine.

In other embodiments, the disclosed methods include comparing one or more indicators of diabetes (such as glucose tolerance, triglyceride levels, free fatty acid levels, or HbA1c levels) to a control (such as no administration of any of insulin, any mutated FGF1 protein containing an S116 mutation (such as S116R) (such as a protein generated using the sequences shown in Tables 1 and 2, the sequence in SEQ ID NOS: 11, 13, 14, 15, 16, 17, 18, 19, 20, 21, 22, 23, or 24, or those encoding a protein having at least 90%, at least 92%, at least 95%, at least 96%, at least 97%, at least 98%, at least 99%, or 100% sequence identity to SEQ ID NO: 11, 13, 14, 15, 16, 17, 18, 19, 20, 21, 22, 23, or 24), or a nucleic acid molecule encoding such), wherein an increase or decrease in the particular indicator relative to the control (as discussed above) indicates effective treatment of diabetes. The control can be any suitable control against which to compare the indicator of diabetes in a subject. In some embodiments, the control is a sample obtained from a healthy subject (such as a subject without diabetes). In some embodiments, the control is a historical control or standard reference value or range of values (such as a previously tested control sample, such as a group of subjects with diabetes, or group of samples from subjects that do not have diabetes). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval). In other examples, the control is the subject (or group of subjects) treated with placebo compared to the same subject (or group of subjects) treated with the therapeutic compound in a cross-over study. In further examples, the control is the subject (or group of subjects) prior to treatment.

The disclosure is illustrated by the following non-limiting Examples.

### Example 1

### Preparation of Mutated FGF1 Proteins

Mutated FGF1 proteins can be made using known methods (e.g., see Xia et al., PLoS One. 7(11):e48210, 2012). An example is provided below.

Briefly, a nucleic acid sequence encoding an FGF1 mutant protein (e.g., any of SEQ ID NOS: 11-24) can be fused downstream of an enterokinase (EK) recognition sequence (Asp₄Lys) preceded by a flexible 20 amino acid linker (derived from the S-tag sequence of pBAC-3) and an N-terminal (His)₆ tag. The resulting expressed fusion protein utilizes the (His)₆ tag for efficient purification and can be subsequently processed by EK digestion to yield the mutant FGF1 protein.

The mutant FGF1 protein can be expressed from an E. coli host after induction with isopropyl-β-D-thio-galactoside. The expressed protein can be purified utilizing sequential column chromatography on Ni-nitrilotriacetic acid (NTA) affinity resin followed by ToyoPearl HW-40S size exclusion chromatography. The purified protein can be digested with EK to remove the N-terminal (His)₆ tag, 20 amino acid linker, and (Asp₄Lys) EK recognition sequence. A subsequent second Ni-NTA chromatographic step can be utilized to remove the released N-terminal mutant FGF1 protein (along with any uncleaved fusion protein). Final purification can be performed using HiLoad Superdex 75 size exclusion chromatography equilibrated to 50 mM Na₂PO₄, 100 mM NaCl, 10 mM (NH₄)₂SO₄, 0.1 mM ethylenediaminetetraacetic acid (EDTA), 5 mM L-Methionine, pH at 6.5 (“PBX” buffer); L-Methionine can be included in PBX buffer to limit oxidization of reactive thiols and other potential oxidative degradation.

In some examples, the enterokinase is not used, and instead, an FGF1 mutant protein (such as one that includes an N-terminal methionine) can be made and purified using heparin affinity chromatography.

For storage and use, the purified mutant FGF1 protein can be sterile filtered through a 0.22 micron filter, purged with N2, snap frozen in dry ice and stored at −80° C. prior to use. The purity of the mutant FGF1 protein can be assessed by both Coomassie Brilliant Blue and Silver Stain Plus (BIO-RAD Laboratories, Inc., Hercules Calif.) stained sodium dodecyl sulfate polyacrylamide gel electrophoresis (SDS PAGE). Mutant FGF1 proteins can be prepared in the absence of heparin. Prior to IV bolus, heparin, or PBS, can be added to the protein.

### Example 2

### FGF1 Mutants Containing S116R Mutation Reduce Blood Glucose and Food Intake in Ob/Ob Mice

### Animals

Mice were housed in a temperature-controlled environment with a 12-hour light/12-hour dark cycle and handled according to institutional guidelines complying with U.S. legislation. Male ob/ob mice (B6.V-Lep^{ob}/J, Jackson laboratories) received a standard diet (MI laboratory rodent diet 5001, Harlan Teklad) and acidified water ad libitum. Proteins or vehicle were injected as described.

### Serum Analysis

Blood was collected by tail bleeding either in the ad libitum fed state or following overnight fasting. Serum glucose levels were measured using a OneTouch Ultra glucometer (Lifescan Inc).

### Food Intake

Daily food intake was measured by recording the change in the total cage food weight each day and proportioning the difference equally to the mice in each cage.

### Purification of FGF Proteins

Each of SEQ ID NOS: 11-24 were expressed in Escherichia coli cells and purified from the soluble bacterial cell lysate fraction by heparin affinity, ion exchange, and size exclusion chromatographies.

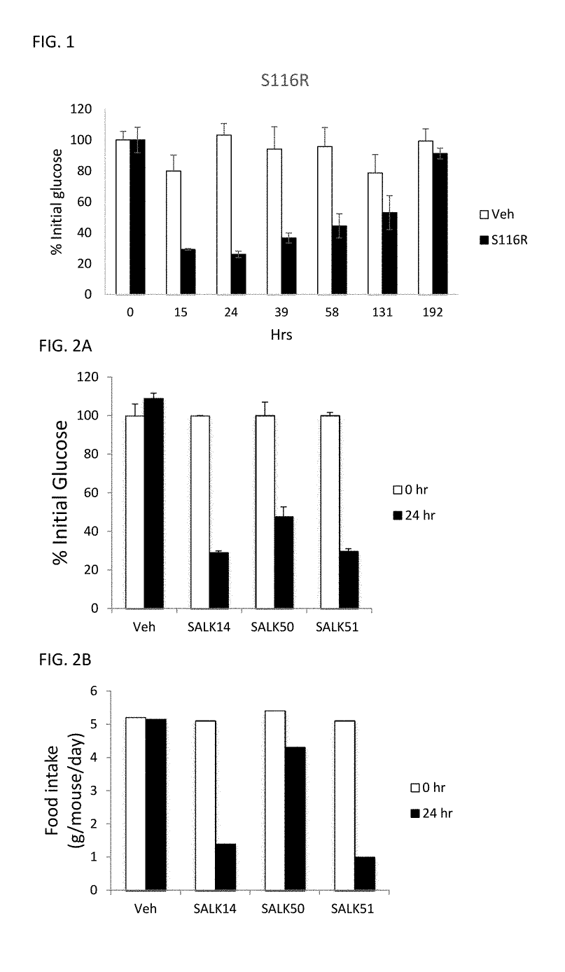

Mice (ob/ob) received a standard diet and acidified water ad libitum. Ob/ob mice were injected subcutaneously with vehicle (PBS) or the FGF1 analog shown in SEQ ID NO: 11 (0.5 mg/kg). Blood glucose levels were monitored at the times shown in FIG. 1. Serum analyses were performed on blood collected by tail bleeding in the ad libitum fed state. As shown in FIG. 1, a single injection of the S116R mutant significantly reduced blood glucose levels, and these effects were observed for a longer duration (up to one week) than seen for SEQ ID NO: 12.

In another experiment, ob/ob mice were injected subcutaneously with vehicle (PBS) or the FGF1 analog shown in SEQ ID NOS: 12, 13, or 14 (0.5 mg/kg) and blood glucose levels and food intake determined. As shown in FIGS. 2A and 2B, the FGF1 mutant proteins significantly reduced blood glucose levels (for example by about 50-80%) and reduced food intake (for example by about 20-80%). The incorporation of S116R did not affect the ability of SEQ ID NO: 12 to lower glucose after 24 hours after injection, or significantly affect the suppression of feeding. The additional inclusion of K12V and N95V mutations were sufficient to dissociate the feeding effects and the glucose lowering effects.

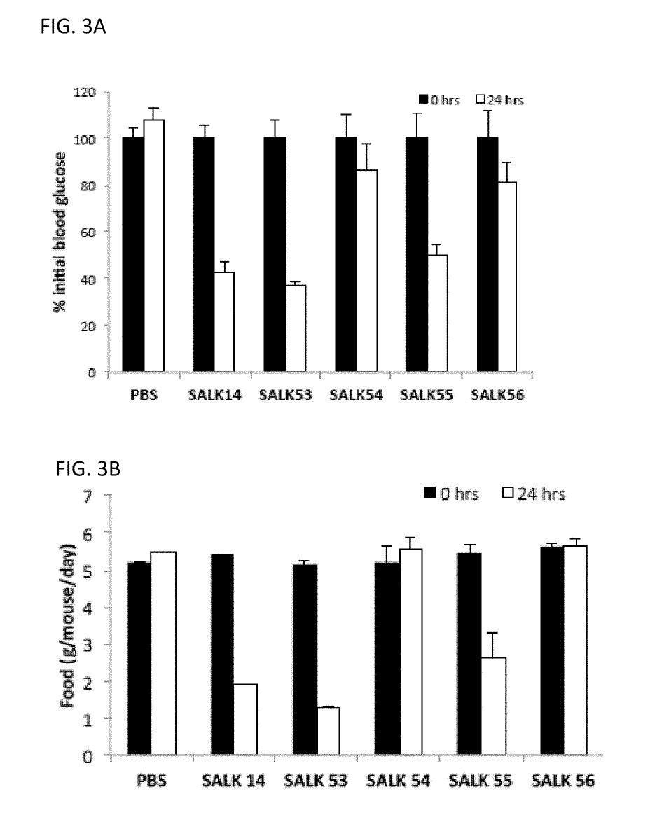

In another experiment, ob/ob mice were injected subcutaneously with vehicle (PBS) or the FGF1 analog shown in SEQ ID NOS: 12, 15, 16, 17, or 18 (0.5 mg/kg) and blood glucose levels and food intake determined. As shown in FIGS. 3A and 3B, several of the FGF1 mutant proteins (such as SEQ ID NOS: 12, 15, and 17) significantly reduced blood glucose levels (for example by about 50-60%) and reduced food intake (for example by about 50-80%). The conservative mutation of N95T does not fully disrupt the interactions with the receptor, compared to N95V, such that the feeding effect of Salk_53 is as pronounced as that seen with Salk_14. The combination of the specific mutations Y55A (SEQ ID NO: 16) and E87H (Salk_056; SEQ ID NO: 18) with the C117V (stabilizing) and S116R (increased heparan sulfate binding) mutations effectively reduce the receptor binding affinity to an extent where efficacy is compromised. The reduced feeding effect is similarly attributed to reduced receptor affinity, mirroring the reduced glucose lowering. Thus while S116R increases heparan sulfate affinity and thereby receptor affinity, this affect cannot compensate for the loss of critical ligand-receptor interactions.

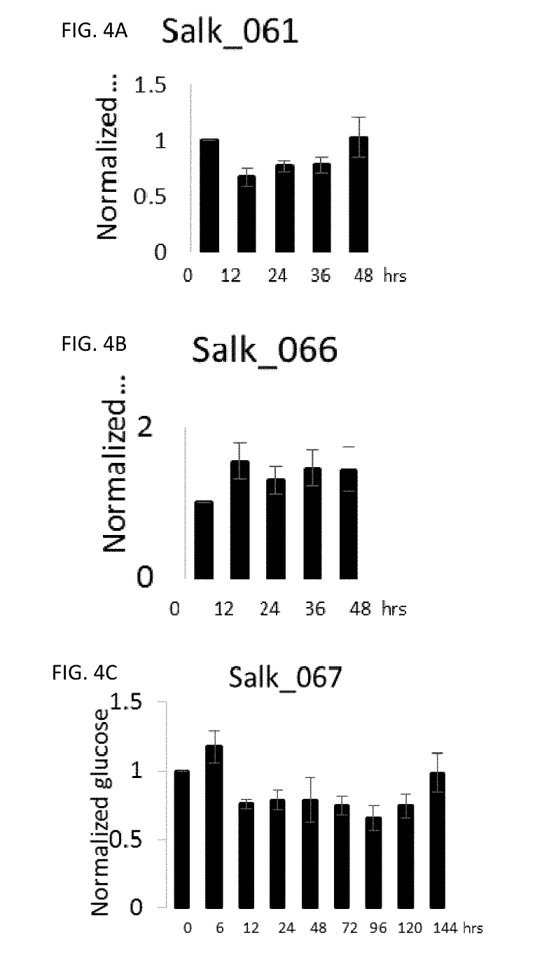

In another experiment, ob/ob mice were injected subcutaneously with vehicle (PBS) or the FGF1 analog shown in SEQ ID NOS: 19, 21 or 22 (0.5 mg/kg) and blood glucose levels determined. As shown in FIGS. 4A-4C, several of the FGF1 mutant proteins (such as SEQ ID NOS: 19 and 2) significantly reduced blood glucose levels (for example by about 20-40%). These results show that mutating three critical ligand-receptor interactions is not tolerated to the extent that the glucose lowering effects are lost. The mutations at K12V, N95V and Y94V were designed to reduce receptor binding affinity. The combination of all three abrogate the glucose effects and the reduced feeding effect is similarly attributed to reduced receptor affinity, mirroring the reduced glucose lowering. Thus while S116R increases heparan sulfate affinity and thereby receptor affinity, this affect cannot compensate for the loss of critical ligand-receptor interactions.

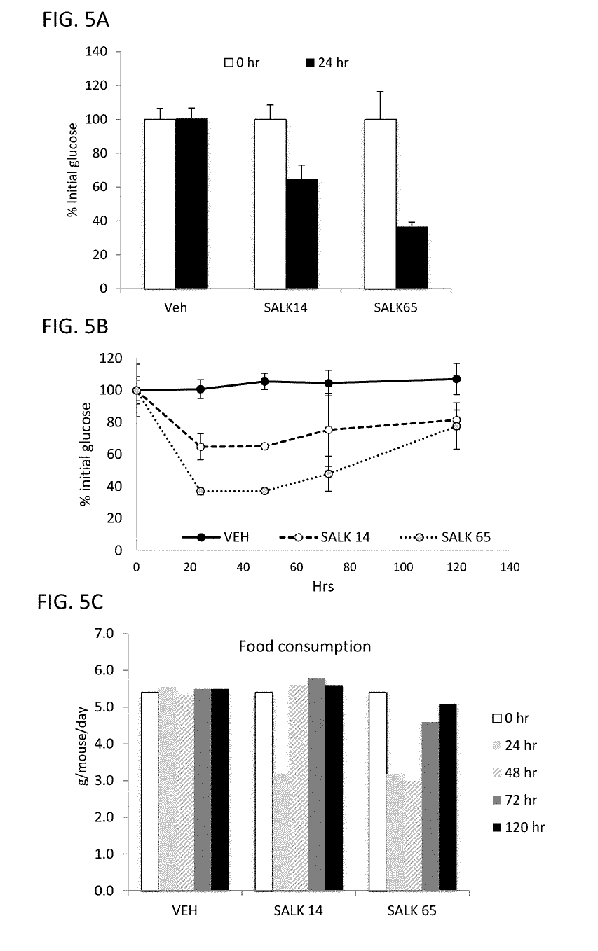

In another experiment, ob/ob mice were injected subcutaneously with vehicle (PBS) or the FGF1 analog shown in SEQ ID NOS: 12 or 20 (0.5 mg/kg) and blood glucose levels and food intake determined. As shown in FIGS. 5A-5C, the FGF1 mutant proteins significantly reduced blood glucose levels (for example by about 40-80%) and reduced food intake (for example by about 30-45%).

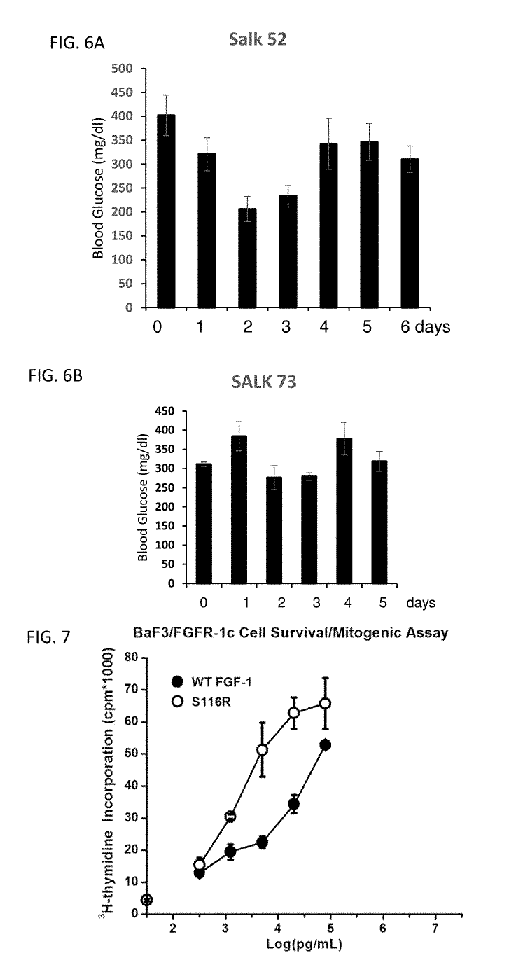

The FGF1 mutant shown in SEQ ID NO: 24 (Salk_052) was mutated to include an artificial disulfide bond between amino acid positions 66 and 83 (SEQ ID NO: 23; Salk_074). As shown in FIGS. 6A and 6B, an artificial disulfide bond between amino acid positions 66 and 83 is not tolerated in SEQ ID NO: 23 (the glucose lowering ability is lost).

### Example 3

### BaF3/FGFR-1c Cell Culture and Cell Survival/Mitogenic Stimulation by FGF-1 and S116R Mutant Protein

A BaF3 cell system expressing FGFR-1c (Ornitz et al., 1996, J Biol Chem 271(25):15292-7) was used to quantify receptor activation by WT FGF-1 and an S116R mutant protein. The BaF3 cells lack membrane-bound HS proteoglycan, and heparin sulfate (1 μg/mL) was added to the assay to promote the ternary FGF-1/FGFR-1c/HS signal transduction complex formation. Cells were maintained in RPMI 1640 media (Sigma Chemical, St. Louis Mo.) supplemented with 10% newborn calf serum (NCS) (Sigma Chemical, St. Louis Mo.), 0.5 ng/mL murine recombinant interleukin-3 (mIL-3, PeproTech Inc., Rocky Hill N.J.), 2 mM L-glutamine, penicillin-streptomycin and 50 μM β-mercaptoethanol (“BaF3 culture medium”), and G418 (600 μg/mL). FGFR-1c expressing BaF3 cells were washed twice in BaF3 “assay media” (“culture media” lacking mIL-3) and plated at a density of 30,000 cells/well in a 96-well assay plate in assay media containing heparin (1 μg/mL) and concentrations of recombinant WT FGF-1 (SEQ ID NO: 5) and S116R mutant protein (SEQ ID NO: 11) ranging from 0.02 to 5 nM (3.18×10²-7.95×10⁴ pg/mL). The cells were incubated for 36 h and mitogenic activity was determined by adding 1 μCi of ³H-thymidine in 50 μL of BaF3 assay medium to each well. Cells were harvested after 4 h by filtration through glass fiber paper. Incorporated ³H-thymidine was counted on a Wallac β plate scintillation counter (PerkinElmer, Waltham Mass.).

The mitogenic/cell survival response of BaF3 cells expressing FGFR-1c towards WT FGF-1 and S116R mutant in the presence of added heparin is shown in FIG. 7. The BaF3/FGFR-1c cell system is more responsive to FGF-1 than the NIH 3T3 fibroblasts, and a mitogenic response is quantified primarily over a concentration range of 2.5-5.0 log pg/mL. The S116R mutant exhibits an increase in mitogenic activity in comparison to WT FGF-1. Over the range of approximately 3-4 log pg/mL the S116R mutant exhibits ˜10× greater mitogenic potency, on an equivalent concentration basis, compared to WT FGF-1.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

We claim:

1. A method of reducing blood glucose in a mammal, comprising:
administering a therapeutically effective amount of a mutated mature fibroblast growth factor (FGF) 1 protein to the mammal, or a nucleic acid molecule encoding the mutated FGF1 protein or a vector comprising the nucleic acid molecule, thereby reducing the blood glucose,
wherein the mutated mature FGF1 protein comprises:
an S116 mutation;
optionally a deletion of at least six contiguous N-terminal amino acids;
optionally at least one additional point mutation;
or combinations thereof.

2. A method of treating a metabolic disease in a mammal, comprising:
administering a therapeutically effective amount of a mutated mature fibroblast growth factor (FGF) 1 protein to the mammal, or a nucleic acid molecule encoding the mutated FGF1 protein or a vector comprising the nucleic acid molecule, thereby treating the metabolic disease,
wherein the mutated mature FGF1 protein comprises:
an S116 mutation;
optionally a deletion of at least six contiguous N-terminal amino acids;
optionally at least one additional point mutation;
or combinations thereof.

3. The method of claim 2, wherein the metabolic disease is type 2 diabetes, non-type 2 diabetes, type 1 diabetes, polycystic ovary syndrome (PCOS), metabolic syndrome (MetS), obesity, non-alcoholic steatohepatitis (NASH), non-alcoholic fatty liver disease (NAFLD), hyperlipidemia, hypertension, latent autoimmune diabetes (LAD), or maturity onset diabetes of the young (MODY).

4. A method of reducing fed and fasting blood glucose, improving insulin sensitivity and glucose tolerance, reducing systemic chronic inflammation, ameliorating hepatic steatosis in a mammal, reducing food intake, or combinations thereof, comprising:
administering a therapeutically effective amount of a mutated mature FGF1 protein to the mammal, or a nucleic acid molecule encoding the mutated FGF1 protein or a vector comprising the nucleic acid molecule, thereby reducing fed and fasting blood glucose, improving insulin sensitivity and glucose tolerance, reducing systemic chronic inflammation, ameliorating hepatic steatosis in a mammal, reducing food intake, or combinations thereof,
wherein the mutated mature FGF1 protein comprises:
an S116 mutation;
optionally a deletion of at least six contiguous N-terminal amino acids;
optionally at least one additional point mutation;
or combinations thereof.

5. The method of claim 1, wherein the S116 mutation is S116R.

6. The method of claim 1, wherein the therapeutically effective amount of the mutated mature FGF1 protein is at least 0.1 mg/kg.

7. The method of claim 1, wherein the administering is subcutaneous, intraperitoneal, intramuscular, or intravenous.

8. The method of claim 1, wherein the mammal is a cat or dog.

9. The method of claim 1, wherein the mammal is a human.

10. The method of claim 1, wherein the mutated mature FGF1 protein comprises a deletion of at least 9, at least 10, at least 11, at least 12 or at least 13 contiguous N-terminal amino acids, wherein the mutated FGF1 protein has reduced mitogenic activity as compared to a wild-type mature FGF1 protein.

11. The method of claim 1, wherein the at least one additional point mutation comprises a mutation at one or more of K9, K10, K12, L14, Y15, C16, H21, R35, Q40, L44, L46, S47, E49, Y55, M67, L73, C83, L86, E87, H93, Y94, N95, H102, A103, E104, K105, N106, F108, V109, L111, K112, K113, C117, K118, R119, G120, P121, R122, F132, L133, P134, L135, wherein the numbering refers to the sequence shown SEQ ID NO: 5, and wherein the mutated FGF1 protein has reduced mitogenic activity as compared to wild-type mature FGF1 protein.

12. The method of claim 1, wherein the at least one additional point mutation comprises one or more of the mutations shown in Table 1, wherein the mutated FGF1 protein has reduced mitogenic activity as compared to wild-type mature FGF1 protein.

13. The method of claim 1, wherein the at least one additional point mutation comprises mutations at one or more of K12, R35, E49, Y55, E87, Y94, N95, and C117, wherein the numbering refers to SEQ ID NO: 5.

14. The method of claim 10, wherein the wild-type mature FGF1 protein comprises SEQ ID NO: 5.

15. The method of claim 1, wherein the mutated mature FGF1 protein comprises at least 95% sequence identity to SEQ ID NO: 11, 13, 14, 15, 16, 17, 18, 19, 20, 21, 22, 23 or 24.

16. The method of claim 1, wherein the mutated mature FGF1 protein is a chimeric protein.

17. The method of claim 1, wherein the method further comprises administering an additional therapeutic compound.

18. The method of claim 17, wherein the additional therapeutic compound is insulin, an alpha-glucosidase inhibitor, amylin agonist, dipeptidyl-peptidase 4 (DPP-4) inhibitor, meglitinide, sulfonylurea, or a peroxisome proliferator-activated receptor (PPAR)-gamma agonist.

19. The method of claim 18, wherein the PPAR-gamma agonist is a thiazolidinedione (TZD), aleglitazar, farglitazar, muraglitazar, or tesaglitazar.

20. The method of claim 19, wherein the TZD is pioglitazone, rosiglitazone, rivoglitazone, or troglitazone.


SEQUENCE LISTING (82 sequences)

SEQ ID NO: 1 - DNA, 468 bp, Homo sapiens
atggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaa

SEQ ID NO: 2 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 3 - DNA, 468 bp, Mus musculus
atggctgaaggggagatcacaaccttcgcagccctgaccgagaggttcaacctgcctctaggaaactacaaaaagcccaaactgctctactgcagcaacgggggccacttcttgaggatccttcctgatggcaccgtggatgggacaagggacaggagcgaccagcacattcagctgcagctcagtgcggaaagtgcgggcgaagtgtatataaagggtacggagaccggccagtacttggccatggacaccgaagggcttttatacggctcgcagacaccaaatgaggaatgtctgttcctggaaaggctggaagaaaaccattataacacttacacctccaagaagcatgcggagaagaactggtttgtgggcctcaagaagaacgggagctgtaagcgcggtcctcggactcactatggccagaaagccatcttgtttctgcccctcccggtgtcttctgactag

SEQ ID NO: 4 - protein, 155 aa, Mus musculus
MAEGEITTFAALTERFNLPLGNYKKPKLLYCSNGGHFLRILPDGTVDGTRDRSDQHIQLQLSAESAGEVYIKGTETGQYLAMDTEGLLYGSQTPNEECLFLERLEENHYNTYTSKKHAEKNWFVGLKKNGSCKRGPRTHYGQKAILFLPLPVSSD

SEQ ID NO: 5 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 6 - protein, 137 aa, Artificial Sequence
  Synthetic polypeptide
PPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 7 - protein, 131 aa, Artificial sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 8 - protein, 127 aa, Artificial sequence
  Synthetic polypeptide
LYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 9 - protein, 129 aa, Artificial sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 10 - DNA, 414 bp, Artificial sequence
  Synthetic polynucleotide
ccgccgggtaactacaaaaaaccgaaactgctgtattgcagcaacggcggtcattttctgcgtattctgccggatggcaccgtcgacggtacgcgtgatcgcagtgaccagcacattcagctgcaactgagcgcggaatctgtgggtgaagtttatatcaaatcaaccgaaacgggccagtacctggccatggataccgacggcctgctgtacggttcgcaaacgccgaatgaagaatgcctgtttctggaacgtctggaagaaaaccattacaacacctacatcagtaaaaaacacgcggagaaaaactggttcgttggcctgaagaaaaacggttcctgtaaacgcggcccgcgcacccattacggtcaaaaagccattctgtttctgccgctgccggtttcgtccgactaa

SEQ ID NO: 11 - protein, 140 aa, Artificial sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGRCKRGPRTHYGQKAILFLPLPVSSD

SEQ ID NO: 12 - protein, 140 aa, Artificial sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 13 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGRVKRGPRTHYGQKAILFLPLPVSSD

SEQ ID NO: 14 - protein, 131 aa, Artificial sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGRVKRGPRTHYGQKAILFLPLPVSSD

SEQ ID NO: 15 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYTTYISKKHAEKNWFVGLKKNGRVKRGPRTHYGQKAILFLPLPVSSD

SEQ ID NO: 16 - protein, 140 aa, Artificial sequence
  Synthetic polypeptide
FNLPPGNYKKPKLLYCSNGGHFLRILPDGTVDGTRDRSDQHIQLQLSAESVGEVAIKSTETGQYLAMDTDGLLYGSQTPNEECLFLERLEENHYNTYISKKHAEKNWFVGLKKNGRVKRGPRTHYGQKAILFLPLPVSSD

SEQ ID NO: 17 - protein, 140 aa, Artificial sequence
  Synthetic polypeptide
FNLPPGNYKKPKLLYCSNGGHFLRILPDGTVDGTRDRSDQHIQLQLSAESVGEVWIKSTETGQYLAMDTDGLLYGSQTPNEECLFLERLEENHYNTYISKKHAEKNWFVGLKKNGRVKRGPRTHYGQKAILFLPLPVSSD

SEQ ID NO: 18 - protein, 140 aa, Artificial sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLHRLEENHYNTYISKKHAEKNWFVGLKKNGRVKRGPRTHYGQKAILFLPLPVSSD

SEQ ID NO: 19 - protein, 131 aa, Artificial sequence
  Synthetic polypeptide
KPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGRVKRGPRTHYGQKAILFLPLPVSSD

SEQ ID NO: 20 - protein, 140 aa, Artificial sequence
  Synthetic polypeptide
FNLPPGNYKKPKLLYCSNGGHFLRILPDGTVDGTRDRSDQHIQLQLSAASVGEVYIKSTETGQYLAMDTDGLLYGSQTPNEECLFLERLEENHYNTYISKKHAEKNWFVGLKKNGRVKRGPRTHYGQKAILFLPLPVSSD

SEQ ID NO: 21 - protein, 131 aa, Artificial sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHVVTYISKKHAEKNWFVGLKKNGRVKRGPRTHYGQKAILFLPLPVSSD

SEQ ID NO: 22 - protein, 131 aa, Artificial sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGRVKRGPRTHYGQKAILFLPLPVSSD

SEQ ID NO: 23 - protein, 131 aa, Artificial sequence
  Synthetic polypeptide
KPKLLYCSNGGHFLRILPDGTVDGTRDRSDQHIQLQLSAESVGEVYIKSTETGQYLCMDTDGLLYGSQTPNEECLFLERLEENHYVTYISKKHAEKNWFVGLKKNGRVKRGPRTHYGQKAILFLPLPVSSD

SEQ ID NO: 24 - protein, 131 aa, Artificial sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGRVKRGPRTHYGQKAILFLPLPVSSD

SEQ ID NO: 25 - protein, 42 aa, Artificial sequence
  Synthetic polypeptide
PGLPPALPEPPGILAPQPPDVGSSDPLSMVGPSQGRSPSYAS

SEQ ID NO: 26 - protein, 48 aa, Artificial sequence
  Synthetic polypeptide
LPMVPEEPEDLRGHLESDMFSSPLETDSMDPFGLVTGLEAVRSPSFEK

SEQ ID NO: 27 - protein, 90 aa, Artificial sequence
  Synthetic polypeptide
CGADQFRCGNGSCVPRAWRCDGVDDCGDGSDEAPEICETPTCQSNEFRCRSGRCIPQHWLCDGLNDCGDGSDESQQCSAPASEPPGSLSL

SEQ ID NO: 28 - protein, 37 aa, Artificial sequence
  Synthetic polypeptide
CGADQFRCGNGSCVPRAWRCDGVDDCGDGSDEAPEIC

SEQ ID NO: 29 - protein, 37 aa, Artificial Sequence
  Synthetic polypeptide
  MISC_FEATURE (33)..(33): Xaa = Pro or Ser
  MISC_FEATURE (34)..(34): Xaa = Pro or Gln
  MISC_FEATURE (35)..(35): Xaa = Ala or Gln
  MISC_FEATURE (36)..(36): Xaa = His or no amino acid
CQSNEFRCRSGRCIPQHWLCDGLNDCGDGSDEXXXXC

SEQ ID NO: 30 - protein, 38 aa, Artificial Sequence
  Synthetic polypeptide
  MISC_FEATURE (2)..(2): Xaa = Glu, Leu, Pro, Gln or Arg
  MISC_FEATURE (3)..(3): Xaa = Ala, Pro or Ser
  MISC_FEATURE (4)..(4): Xaa = Asp, Gly, Ile or Asn
  MISC_FEATURE (5)..(5): Xaa = Glu, Gly or Gln
  MISC_FEATURE (6)..(6): Xaa = Phe or Gln
  MISC_FEATURE (7)..(7): Xaa = Phe, Lys, Pro, Gln, Arg or Thr
  MISC_FEATURE (8)..(8): Xaa = Glu or no amino acid
  MISC_FEATURE (10)..(10): Xaa = Gly, Asn, Arg or Ser
  MISC_FEATURE (11)..(11): Xaa = Asn or Ser
  MISC_FEATURE (13)..(13): Xaa = His, Asn, Gln or Arg
  MISC_FEATURE (15)..(15): Xaa = Ile or Val
  MISC_FEATURE (17)..(17): Xaa = Ala, Glu, Leu, Pro, Gln, Arg or Val
  MISC_FEATURE (18)..(18): Xaa = Ala, His, Pro, Gln Arg or Thr
  MISC_FEATURE (20)..(20): Xaa = Leu, Arg or Val
  MISC_FEATURE (24)..(24): Xaa = Asp, Glu or Val
  MISC_FEATURE (25)..(25): Xaa = Asp, Asn or Pro
  MISC_FEATURE (28)..(28): Xaa = Gly, Leu or Gln
  MISC_FEATURE (30)..(30): Xaa = Asp, Gly, Asn or Ser
  MISC_FEATURE (34)..(34): Xaa = Ala, Glu, Lys or Thr
  MISC_FEATURE (35)..(35): Xaa = Asp, Gly, Leu, Asn or Ser
  MISC_FEATURE (36)..(36): Xaa = Ala or no amino acid
  MISC_FEATURE (37)..(37): Xaa = His or no amino acid
CXXXXXXXCXXGXCXPXXWXCDGXXDCXDXSDEXXXXC

SEQ ID NO: 31 - protein, 38 aa, Artificial Sequence
  Synthetic polypeptide
  MISC_FEATURE (2)..(2): Xaa = Ala, Gly or Pro
  MISC_FEATURE (3)..(3): Xaa = Ala, Pro or Ser
  MISC_FEATURE (4)..(4): Xaa = Asp, Gly or Asn
  MISC_FEATURE (5)..(5): Xaa = Glu or Gln
  MISC_FEATURE (7)..(7): Xaa = Gln, Arg or Thr
  MISC_FEATURE (9)..(9): Xaa = Gly, Gln, Arg or Ser
  MISC_FEATURE (10)..(10): Xaa = Gly, Gln or Ser
  MISC_FEATURE (11)..(11): Xaa = Gly or Thr
  MISC_FEATURE (12)..(12): Xaa = Gly, Lys, Gln or Ser
  MISC_FEATURE (13)..(13): Xaa = Ile, Lys or no amino acid
  MISC_FEATURE (15)..(15): Xaa = Ile, Leu or Val
  MISC_FEATURE (17)..(17): Xaa = Leu, Gln, Arg or Val
  MISC_FEATURE (18)..(18): Xaa = Ala, Glu, His or Pro
  MISC_FEATURE (20)..(20): Xaa = Leu, Arg or Val
  MISC_FEATURE (24)..(24): Xaa = Asp, Leu or Val
  MISC_FEATURE (25)..(25): Xaa = Asp or Asn
  MISC_FEATURE (30)..(30): Xaa = Gly or Asn
  MISC_FEATURE (34)..(34): Xaa = Ala, Glu, Lys, Pro or Ser
  MISC_FEATURE (35)..(35): Xaa = Gly, Leu, Pro or Ser
  MISC_FEATURE (36)..(36): Xaa = Ala, Glu, Val or no amino acid
  MISC_FEATURE (37)..(37): Xaa = Ile, Thr or no amino acid
CXXXXFXCXXXXXCXPXXWXCDGXXDCGDXSDEXXXXC

SEQ ID NO: 32 - protein, 47 aa, Artificial Sequence
  Synthetic polypeptide
CQSNEFRCRSGRCIPQHWLCDGLNDCGDGSDESQQCSAPASEPPGSL

SEQ ID NO: 33 - protein, 36 aa, Artificial Sequence
  Synthetic polypeptide
CQSNEFRCRSGRCIPQHWLCDGLNDCGDGSDESQQC

SEQ ID NO: 34 - protein, 35 aa, Artificial Sequence
  Synthetic polypeptide
CRAGEFRCSNGRCVPLTWLCDGEDDCQDNSDEKNC

SEQ ID NO: 35 - protein, 38 aa, Artificial Sequence
  Synthetic polypeptide
CPSNQFPCRSTGICIPLAWVCDGLNDCGDGSDESPAHC

SEQ ID NO: 36 - protein, 37 aa, Artificial Sequence
  Synthetic polypeptide
CQSNEFRCRSGRCIPQHWLCDGLNDCGDGSDEPPAHC

SEQ ID NO: 37 - protein, 37 aa, Artificial Sequence
  Synthetic polypeptide
  MISC_FEATURE (9)..(9): Xaa = Ser or Gly
  MISC_FEATURE (17)..(17): Xaa = His or Thr
  MISC_FEATURE (19)..(19): Xaa = Val or Leu
  MISC_FEATURE (33)..(33): Xaa = Pro or Ser
  MISC_FEATURE (36)..(36): Xaa = His or Thr
CLPDEFQCXSGRCIPQXWXCDGLNDCGDGSDEXPAXC

SEQ ID NO: 38 - protein, 38 aa, Artificial Sequence
  Synthetic polypeptide
  MISC_FEATURE (2)..(2): Xaa = Ala, Gly, Pro or Arg
  MISC_FEATURE (3)..(3): Xaa = Ala or Pro
  MISC_FEATURE (4)..(4): Xaa = Asp, Gly, Asn or Ser
  MISC_FEATURE (5)..(5): Xaa = Glu or Gly
  MISC_FEATURE (7)..(7): Xaa = Gln, Arg or Thr
  MISC_FEATURE (9)..(9): Xaa = Gly, Ser or Lys
  MISC_FEATURE (11)..(11): Xaa = Gly or Thr
  MISC_FEATURE (12)..(12): Xaa = Gly, His or Arg
  MISC_FEATURE (13)..(13): Xaa = Arg or no amino acid
  MISC_FEATURE (15)..(15): Xaa = Ile, Leu or Val
  MISC_FEATURE (16)..(16): Xaa = Pro or Ser
  MISC_FEATURE (17)..(17): Xaa = Ala, Leu or Gln
  MISC_FEATURE (18)..(18): Xaa = Asn, Ser or Thr
  MISC_FEATURE (20)..(20): Xaa = Leu, Arg or Val
  MISC_FEATURE (24)..(24): Xaa = Asp, Glu or Val
  MISC_FEATURE (28)..(28): Xaa = Gly or Leu
  MISC_FEATURE (34)..(34): Xaa = Ala, Glu or Thr
  MISC_FEATURE (35)..(35): Xaa = Asp, Asn or Ser
  MISC_FEATURE (36)..(36): Xaa = Ala or no amino acid
  MISC_FEATURE (37)..(37): Xaa = Thr or no amino acid
CXXXXFXCXNXXXCXXXXWXCDGXDDCXDGSDEXXXXC

SEQ ID NO: 39 - protein, 37 aa, Artificial Sequence
  Synthetic polypeptide
CGPDQFRCSSGKCIPQHWLCDGLNDCGDGSDESPATC

SEQ ID NO: 40 - protein, 37 aa, Artificial Sequence
  Synthetic polypeptide
CGANEFQCRSTGICVPVEWVCDGDNDCGDGSDEPPVC

SEQ ID NO: 41 - protein, 37 aa, Artificial Sequence; identical to SEQ ID NO: 28
  Synthetic polypeptide

SEQ ID NO: 42 - protein, 37 aa, Artificial Sequence
  Synthetic polypeptide
CGPDEFRCNNGQCIPLPWRCDGVDDCGDNSDEPLEIC

SEQ ID NO: 43 - protein, 35 aa, Artificial Sequence
  Synthetic polypeptide
CASGEFTCNNGQCVPLAWRCDGVNDCQDGSDEKGC

SEQ ID NO: 44 - protein, 36 aa, Artificial Sequence
  Synthetic polypeptide
CPPDEFQCRGTKKCLPLAWVCDGDNDCEDDSDEESC

SEQ ID NO: 45 - protein, 37 aa, Artificial Sequence
  Synthetic polypeptide
CLPDEFQCGSGRCIPQHWLCDGLNDCGDGSDEPPAHC

SEQ ID NO: 46 - protein, 36 aa, Artificial Sequence
  Synthetic polypeptide
CAPGEFTCKNTGRCIPLNWRCDGDDDCGDGSDETDC

SEQ ID NO: 47 - protein, 29 aa, Artificial Sequence
  Synthetic polypeptide
  MISC_FEATURE (2)..(2): Xaa = Ala, Gly or Pro
  MISC_FEATURE (3)..(3): Xaa = Ala, Pro or Ser
  MISC_FEATURE (4)..(4): Xaa = Asp, Gly or Asn
  MISC_FEATURE (5)..(5): Xaa = Glu or Gln
  MISC_FEATURE (7)..(7): Xaa = Gln, Arg or Thr
  MISC_FEATURE (9)..(9): Xaa = Gly, Asn, Arg or Ser
  MISC_FEATURE (10)..(10): Xaa = Gly, Asn or Ser
  MISC_FEATURE (11)..(11): Xaa = Gly or Thr
  MISC_FEATURE (12)..(12): Xaa = Gly, Lys, Gln or Ser
  MISC_FEATURE (13)..(13): Xaa = Ile, Lys or no amino acid
  MISC_FEATURE (15)..(15): Xaa = Ile, Leu or Val
  MISC_FEATURE (17)..(17): Xaa = Leu, Gln, Arg or Val
  MISC_FEATURE (18)..(18): Xaa = Ala, Glu, His or Pro
  MISC_FEATURE (20)..(20): Xaa = Leu, Arg or Val
  MISC_FEATURE (24)..(24): Xaa = Asp, Leu or Val
  MISC_FEATURE (25)..(25): Xaa = Asp or Asn
CXXXXFXCXXXXXCXPXXWXCDGXXDCGD

SEQ ID NO: 48 - protein, 48 aa, Artificial Sequence
  Synthetic polypeptide
CGADQFRCGNGSCVPRAWRCDGVDDCGDGSDEAPEICETPTCQSNEFR

SEQ ID NO: 49 - protein, 40 aa, Artificial Sequence
  Synthetic polypeptide
CRSGRCIPQHWLCDGLNDCGDGSDESQQCSAPASEPPGSL

SEQ ID NO: 50 - protein, 38 aa, Artificial Sequence; identical to SEQ ID NO: 38
  Synthetic polypeptide
  MISC_FEATURE (2)..(2): Xaa = Ala, Gly, Pro or Arg
  MISC_FEATURE (3)..(3): Xaa = Ala or Pro
  MISC_FEATURE (4)..(4): Xaa = Asp, Gly, Asn or Ser
  MISC_FEATURE (5)..(5): Xaa = Glu or Gln
  MISC_FEATURE (7)..(7): Xaa = Gln, Arg or Thr
  MISC_FEATURE (9)..(9): Xaa = Gly, Ser or Lys
  MISC_FEATURE (11)..(11): Xaa = Gly or Thr
  MISC_FEATURE (12)..(12): Xaa = Gly, His or Arg
  MISC_FEATURE (13)..(13): Xaa = Arg or no amino acid
  MISC_FEATURE (15)..(15): Xaa = Ile, Leu or Val
  MISC_FEATURE (16)..(16): Xaa = Pro or Ser
  MISC_FEATURE (17)..(17): Xaa = Ala, Leu or Gln
  MISC_FEATURE (18)..(18): Xaa = Asn, Ser or Thr
  MISC_FEATURE (20)..(20): Xaa = Leu, Arg or Val
  MISC_FEATURE (24)..(24): Xaa = Asp, Glu or Val
  MISC_FEATURE (28)..(28): Xaa = Gly or Leu
  MISC_FEATURE (34)..(34): Xaa = Ala, Glu or Thr
  MISC_FEATURE (35)..(35): Xaa = Asp, Asn or Ser
  MISC_FEATURE (36)..(36): Xaa = Ala or no amino acid
  MISC_FEATURE (37)..(37): Xaa = Thr or no amino acid

SEQ ID NO: 51 - protein, 38 aa, Artificial Sequence; identical to SEQ ID NO: 30
  Synthetic polypeptide
  MISC_FEATURE (2)..(2): Xaa = Glu, Lue, Pro Gln or Arg
  MISC_FEATURE (3)..(3): Xaa = Ala, Pro or Ser
  MISC_FEATURE (4)..(4): Xaa = Asp, Gly, Ile or Asn
  MISC_FEATURE (5)..(5): Xaa = Glu, Gly or Gln
  MISC_FEATURE (6)..(6): Xaa = Phe or Gln
  MISC_FEATURE (7)..(7): Xaa = Phe, Lys, Pro, Gln, Arg or Thr
  MISC_FEATURE (8)..(8): Xaa = Glu or no amino acid
  MISC_FEATURE (10)..(10): Xaa = Gly, Asn, Arg or Ser
  MISC_FEATURE (11)..(11): Xaa = Asn or Ser
  MISC_FEATURE (13)..(13): Xaa = His, Asn, Gln or Arg
  MISC_FEATURE (15)..(15): Xaa = Ile or Val
  MISC_FEATURE (17)..(17): Xaa = Ala, Glu, Leu, Pro, Gln, Arg or Val
  MISC_FEATURE (18)..(18): Xaa = Ala, His, Pro, Gln, Arg or Thr
  MISC_FEATURE (20)..(20): Xaa = Leu, Arg or Val
  MISC_FEATURE (24)..(24): Xaa = Asp, Glu or Gly
  MISC_FEATURE (25)..(25): Xaa = Asp, Asn or Pro
  MISC_FEATURE (28)..(28): Xaa = Gly, Leu or Gln
  MISC_FEATURE (30)..(30): Xaa = Asp, Gly, Asn or Ser
  MISC_FEATURE (34)..(34): Xaa = Ala, Glu, Lys or Thr
  MISC_FEATURE (35)..(35): Xaa = Asp, Gly, Leu, Asn or Ser
  MISC_FEATURE (36)..(36): Xaa = Ala or no amino acid
  MISC_FEATURE (37)..(37): Xaa = His or no amino acid

SEQ ID NO: 52 - protein, 38 aa, Artificial Sequence; identical to SEQ ID NO: 31
  Synthetic polypeptide
  MISC_FEATURE (2)..(2): Xaa = Ala, Gly or Pro
  MISC_FEATURE (3)..(3): Xaa = Ala, Pro or Ser
  MISC_FEATURE (4)..(4): Xaa = Asp, Gly or Asn
  MISC_FEATURE (5)..(5): Xaa = Glu or Gln
  MISC_FEATURE (7)..(7): Xaa = Gln, Arg or Thr
  MISC_FEATURE (9)..(9): Xaa = Gly, Asn, Arg or Ser
  MISC_FEATURE (10)..(10): Xaa = Gly, Asn or Ser
  MISC_FEATURE (11)..(11): Xaa = Gly or Thr
  MISC_FEATURE (12)..(12): Xaa = Gly, Lys, Gln or Ser
  MISC_FEATURE (13)..(13): Xaa = Ile, Lys or no amino acid
  MISC_FEATURE (15)..(15): Xaa = Ile, Leu or Val
  MISC_FEATURE (17)..(17): Xaa = Leu, Gln, Arg or Val
  MISC_FEATURE (18)..(18): Xaa = Ala, Glu, His or Pro
  MISC_FEATURE (20)..(20): Xaa = Leu, Arg or Val
  MISC_FEATURE (24)..(24): Xaa = Asp, Leu or Val
  MISC_FEATURE (25)..(25): Xaa = Asp or Asn
  MISC_FEATURE (30)..(30): Xaa = Gly or Asn
  MISC_FEATURE (34)..(34): Xaa = Ala, Glu, Lys, Pro or Ser
  MISC_FEATURE (35)..(35): Xaa = Gly, Leu, Pro or Ser
  MISC_FEATURE (36)..(36): Xaa = Ala, Glu, Val or no amino acid
  MISC_FEATURE (37)..(37): Xaa = Ile, Thr or no amino acid

SEQ ID NO: 53 - protein, 36 aa, Artificial Sequence
  Synthetic polypeptide
  MISC_FEATURE (3)..(3): Xaa = Ala or Glu
  MISC_FEATURE (4)..(4): Xaa = Gly or Ser
  MISC_FEATURE (9)..(9): Xaa = Gly or Arg
  MISC_FEATURE (10)..(10): Xaa = Asn, Arg or Ser
  MISC_FEATURE (11)..(11): Xaa = Ala, Ser or Thr
  MISC_FEATURE (12)..(12): Xaa = Lys or Asn
  MISC_FEATURE (17)..(17): Xaa = Glu, His, Gln or Ser
  MISC_FEATURE (18)..(18): Xaa = Ala or Val
  MISC_FEATURE (20)..(20): Xaa = Ile or Val
  MISC_FEATURE (24)..(24): Xaa = Ile or Val
  MISC_FEATURE (28)..(28): Xaa = Asp or Glu
  MISC_FEATURE (34)..(34): Xaa = Asp, Lys, Met, Asn or Thr
  MISC_FEATURE (35)..(35): Xaa = Asn, Ser or Tyr
CGXXLFTCXXXXICISXXWXCDGXDDCXDNSDEXXC

SEQ ID NO: 54 - protein, 47 aa, Artificial sequence
  Synthetic polypeptide
CGEGLFTCRSTNICISHAWVCDGVDDCEDNSDENNCSAPASEPPGSL

SEQ ID NO: 55 - protein, 36 aa, Artificial sequence
  Synthetic polypeptide
CGAGLFTCRSTNICISQVWVCDGVDDCEDNSDEDSC

SEQ ID NO: 56 - protein, 36 aa, Artificial sequence
  Synthetic polypeptide
CGAGLFTCRSTNICISQAWVCDGVDDCEDNSDENYC

SEQ ID NO: 57 - protein, 36 aa, Artificial sequence
  Synthetic polypeptide
CGAGLFTCRSTNICISQAWVCDGVDDCEDNSDETNC

SEQ ID NO: 58 - protein, 36 aa, Artificial sequence
  Synthetic polypeptide
CGEGLFTCGSTNICISSAWVCDGVDDCEDNSDENNC

SEQ ID NO: 59 - protein, 36 aa, Artificial sequence
  Synthetic polypeptide
CGEGLFTCRSTNICISHAWVCDGVDDCEDNSDENNC

SEQ ID NO: 60 - protein, 36 aa, Artificial sequence
  Synthetic polypeptide
CGEGLFTCRSTNICISEAWICDGVDDCEDNSDEKNC

SEQ ID NO: 61 - protein, 36 aa, Artificial sequence
  Synthetic polypeptide
CGAGLFTCRSAKICISHAWVCDGIDDCEDNSDENNC

SEQ ID NO: 62 - protein, 36 aa, Artificial sequence
  Synthetic polypeptide
CGAGLFTCRNSKICISQAWVCDGVDDCDDNSDEKYC

SEQ ID NO: 63 - protein, 36 aa, Artificial sequence
  Synthetic polypeptide
CGASLFTCRRSNICISQAWVCDGVDDCEDNSDEMNC

SEQ ID NO: 64 - protein, 36 aa, Artificial sequence
  Synthetic polypeptide
CGAGLFTCRSTKICISQAWVCDGVDDCEDNSDEKNC

SEQ ID NO: 65 - protein, 47 aa, Artificial sequence
  Synthetic polypeptide
CGAGLFTCRSTNICISQVWVCDGVDDCEDNSDEDSCSAPASEPPGSL

SEQ ID NO: 66 - protein, 47 aa, Artificial sequence
  Synthetic polypeptide
CGAGLFTCRSTNICISQAWVCDGVDDCEDNSDENYCSAPASEPPGSL

SEQ ID NO: 67 - protein, 47 aa, Artificial sequence
  Synthetic polypeptide
CGAGLFTCRSTNICISQAWVCDGVDDCEDNSDETNCSAPASEPPGSL

SEQ ID NO: 68 - protein, 47 aa, Artificial sequence
  Synthetic polypeptide
CGEGLFTCGSTNICISSAWVCDGVDDCEDNSDENNCSAPASEPPGSL

SEQ ID NO: 69 - protein, 47 aa, Artificial sequence
  Synthetic polypeptide
CGEGLFTCRSTNICISEAWICDGVDDCEDNSDEKNCSAPASEPPGSL

SEQ ID NO: 70 - protein, 47 aa, Artificial sequence
  Synthetic polypeptide
CGAGLFTCRSAKICISHAWVCDGIDDCEDNSDENNCSAPASEPPGSL

SEQ ID NO: 71 - protein, 47 aa, Artificial sequence
  Synthetic polypeptide
CGAGLFTCRNSKICISQAWVCDGVDDCDDNSDEKYCSAPASEPPGSL

SEQ ID NO: 72 - protein, 47 aa, Artificial sequence
  Synthetic polypeptide
CGASLFTCRRSNICISQAWVCDGVDDCEDNSDEMNCSAPASEPPGSL

SEQ ID NO: 73 - protein, 47 aa, Artificial sequence
  Synthetic polypeptide
CGAGLFTCRSTKICISQAWVCDGVDDCEDNSDEKNCSAPASEPPGSL

SEQ ID NO: 74 - protein, 135 aa, Artificial sequence
  Synthetic polypeptide
CGADQFRCGNGSCVPRAWRCDGVDDCGDGSDEAPEICETPTCQSNEFRCRSGRCIPQHWLCDGLNDCGDGSDESQQCSAPASEPPGSLCGEGLFTCRSTNICISHAWVCDGVDDCEDNSDENNCSAPASEPPGSL

SEQ ID NO: 75 - protein, 134 aa, Artificial sequence
  Synthetic polypeptide
CLPDEFQCGSGRCIPQHWLCDGLNDCGDGSDEPPAHCSAPASEPPGSLCRAGEFRCSNGRCVPLTWLCDGEDDCQDNSDEKNCAQPTCGEGLFTCRSTNICISHAWVCDGVDDCEDNSDENNCSAPASEPPGSL

SEQ ID NO: 76 - protein, 136 aa, Artificial sequence
  Synthetic polypeptide
CAPGEFTCKNTGRCIPLNWRCDGDDDCGDGSDETDCPAPTCPSNQFPCRSTGICIPLAWVCDGLNDCGDGSDESPAHCSAPASEPPGSLCGEGLFTCRSTNICISHAWVCDGVDDCEDNSDENNCSAPASEPPGSL

SEQ ID NO: 77 - protein, 136 aa, Artificial sequence
  Synthetic polypeptide
CGPDEFRCNNGQCIPLPWRCDGVDDCGDNSDEPLEICQAPTCQSNEFRCRSGRCIPQHWLCDGLNDCGDGSDEPPAHCSAPASEPPGSLCGEGLFTCRSTNICISHAWVCDGVDDCEDNSDENNCSAPASEPPGSL

SEQ ID NO: 78 - protein, 47 aa, Artificial sequence; identical to SEQ ID NO: 54
  Synthetic polypeptide

SEQ ID NO: 79 - protein, 155 aa, Gorilla gorilla; identical to SEQ ID NO: 2

SEQ ID NO: 80 - protein, 155 aa, Pan troglodytes
MAEGEITTFTALTEKFNLPSGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 81 - protein, 155 aa, Canis lupus
MAEGEITTFTALTEKFNLPPGNYMKPKLLYCSNGGHFLRILPDGTVDGTRDRSDQHIQLQLSAESVGEVYIKSTETGQYLAMDTDGLLYGSQTPNEECLFLERLEENHYNTYTSKKHAEKNWFVGLKKNGSCKRGPRTHYGQKAILFLPLPVSSD

SEQ ID NO: 82 - protein, 155 aa, Felis catus
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYTSKKHAEKNWFVGLKKNGSCKRGPRTHYGQKAILFLPLPVSSD